(19) United States
(12) Patent Application Publication
(10) Pub. No.: US 2003/0024012 A1
(43) Pub. Date: Jan. 30, 2003

(54) Gonadotrophins in plants

(75) Inventors: Latifa Abdennebi-Najar, Palaiseau (FR); Hendrikus Antonius C. Bakker, Ede (NL); Hendrik Jan Bosch, Wageningen (NL); Dietmar Dirnberger, Vienna (AT); Jean-Jacques Remy, Marseille (FR); Herta Steinkeller, Vienna (AT); Dirk Franciscus, M. van de Wiel, Harderwijk (NL)

Correspondence Address: BANNER & WITCOFF, LTD., 28 STATE STREET, 28th FLOOR, BOSTON, MA, 02109, US

(73) Assignee: ID-Lelystad, Instituut voor Dierhouderijen Diergezondheid B.V., Lelystad (NL)

(21) Appl. No.: 10/133,205

(22) Filed: Apr. 26, 2002

Related U.S. Application Data
(63) Continuation of application No. PCT/NL00/00774, filed on Oct. 26, 2000.

(30) Foreign Application Priority Data
Oct. 26, 1999 (EP) 99203523.8

(51) Int. Cl. A01H 5/00

(52) U.S. Cl. 800/288; 800/280; 800/317.3

(57) ABSTRACT

The invention relates to the production of gonadotrophins and corresponding receptors in transgenic plants. The invention provides a method to produce a gonadotrophin or its corresponding receptor in a transgenic plant with modified glycosylation machinery, in order to allow for mammalian type of glycosidic side chains of gonadotrophin and its corresponding receptor.

The invention relates to the production of glycoprotein hormones such as thyroid stimulating hormone and gonadotrophins and corresponding receptors in transgenic plants. In animal reproduction the gonadotrophin FSH is employed for superovulation of cattle and for treatment of anestrus in cattle and pigs, whereas LH is employed for treatment of cystic follicles and induction of ovulation. In human medicine FSH together with LH is used to produce eggs for in-vitro fertilization in the treatment of infertility. There is a need for an accessible and standardized source of gonadotrophins such as FSH for therapeutic and diagnostic purposes, which is guaranteed to be free of LH activity.

For example, bovine FSH is difficult to purify in substantial amounts from bovine pituitaries. Previous attempts to produce recombinant bovine FSH have resulted in a product that has been used in clinical trials, but it had little or no bioactivity. A relatively high yield of recombinant bovine FSH (rbFSH) can be obtained in insect cells by the baculovirus expression system, although sufficient upscaling of production has not been achieved yet. Application of (recombinant) gonadotrophin (such as FSH) in the human, for the purpose of assisted reproduction, has become common practice as is evident from the growing number of IVF clinics. Diagnostic methods to monitor treatment effects and to optimize protocols of injections, are routinely applied.

In the human field, TSH and recombinant versions thereof to stimulate thyroid tissue to overcome the need, for elevating endogenous TSH after treatment against thyroid cancer. From a therapeutic perspective, there is considerable interest for the use of novel hTSH analogs. The creation of recombinant proteins as medicaments or pharmaceutical compositions by pharmaco-molecular agriculture constitutes one of the principal attractions of transgenic plants; it is also the domain where their utilization is accepted best by the public opinion. In addition to the yield and the favourable cost which may be expected from the field production of recombinant proteins for therapeutic purposes, transgenic plants present certain advantages over other production systems, such as bacteria, yeasts, and animal cells. Indeed, they are devoid of virus which might be dangerous to humans, and can accumulate the proteins of interest in their “organs of storage”, such as seeds or tubers. This facilitates their handling, their transportation and their storage at ambient temperature, while affording the possibility of subsequent extraction according to needs.

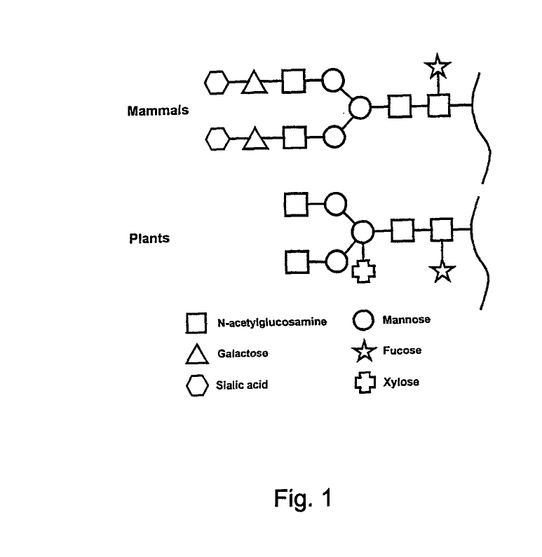

Several heterologous proteins have successfully been produced in plants. Among these proteins are monoclonal antibodies, hormones, vaccine antigens, enzymes and blood proteins (Dieryck et al., 1997; Florack et al., 1995; Ma et al., 1995) Matsumoto et al., 1163; Saito et al., 1991; Thanavala et al. 1995) A major limitation of plants, shared with other heterologous expression systems like bacteria, yeast and insect cells, is their different glycosylation profile compared to mammals. In contrast to bacteria, having no N-linked glycans, and yeast, having only high mannose glycans, plants are able to produce proteins with complex N-linked glycans. Plant glycoproteins have complex N-linked glycans containing a α1,3 linked core fucose and β1,2 linked residues not found in mammals (Lerouge et al., 1998) (FIG. 1). The core of plant N-glycans can, as in mammals, be substituted by 2 GlcNAc¹ residues, which are transferred by N-acetylglucosaminyltransferase I and II (Schachter, 1991) although their appearance varies (Rayon et al., 1999. N-glycans of some plant glycoproteins contain in addition a LewisA (Fuc+1,4(Galβ1,3)GlcNAc) epitope (Fitchette Laine et al., 1997; Melo et al., 1997). However, plant glycoproteins lack the characteristic galactose (NeuAcα2,6Galβ1,4) containing complex N-glycans found in mammals, while also α1,6 linked core fucose is never found (FIG. 1; Schachter, 1991). A mouse monoclonal antibody produced in tobacco plants (Ma et al., 1995) has a typical plant N-glycosylation. 40% High-mannose glycans and 60% complex glycans containing xylose, fucose and 0, 1 or 2 terminal GlcNAc residues (Cabanes Macheteau et al., 1999).

In short, analyses of glycoproteins from plants have indicated that several steps in the glycosylation pathways of plants and mammals are very similar if not identical. There are however also clear differences, particularly in the synthesis of complex glycans. The complex glycans of plants are generally much smaller and contain beta-1,2 xylose or alpha-1,3 fucose residues attached to the Man3 (GlcNAc)2 core. Such residues on glycoprotein are known to be highly immunogenic. This will cause problems for certain applications of recombinant proteins carrying these sugars.

In addition, although common and often essential on mammalian glycoproteins, sialic acid has never been found in plant glycans. This is particularly relevant since experiments with both naturally FSH and recombinant FSH have shown, that the absence of terminal sialic acid on glycosidic side chains can decrease biological activity in vivo. Most likely, asialoglycoprotein-receptors in the liver can bind to asialo-FSH, and thereby cause a rapid clearance of the hormone from the circulation, which is reflected in a reduced metabolic half life and low bioactivity in vivo.

The invention provides a method to produce a glycoprotein hormone such as a thyroid-stimulating hormone or gonadotrophin or its corresponding receptor in a transgenic plant with modified glycosylation machinery, in order to allow for mammalian type of glycosidic side chains of the hormone such as the gonadotrophin and its corresponding receptor. In one embodiment of the invention, tobacco mosaic virus (TMV), the type member of the tobamovirus group of RNA viruses, is used as a viral vector for the expression of these recombinant hormones (in the detailed description gonadotrophins are mainly used) in these transgenic plants. In this expression system it has proven possible to achieve stable high level production of a number of heterologous proteins with desired glycosylation. Glycoprotein hormones, such as FSH, TSH, HCG, HMG, and PMSG, have essentially the alpha subunit in common, whereby the beta subunit effectively determines the specific activity of the hormone, and where here TSH and/or FSH are used, it its clear that also one of the other glycoprotein hormones is applicable .

In particular, the invention provides a method wherein stably transformed tobacco plants with mammalian type of glycosylation are infected with modified TMV in order to produce bioactive rb TSH, rbFSH and rbFSH-R. For expressing recombinant bFSH both subunits of bFSH are inserted separately or together immediately downstream of an additional cp-promoter of TMV and subsequently checked for infectivity. For TSH, analogous methods are for example used, see below. In vitro transcripts are made and both constructs are rubbed mechanically onto the same plants susceptible for TMV. The resulting TMV particles, which can tolerate a larger than wild type genomic RNA can further spread throughout infected plants. Constructs are made which direct proteins into the secretory pathway.

For expression of bovine FSH receptor, a similar approach is used for preparation of the corresponding cDNA homologous oligonucleotide primers will be used to obtain overlapping cDNA fragments. Complete sequences for the receptor are reconstituted in a mammalian expression system. cDNA fragments encoding the N-terminal extracellular domains of the receptors are subcloned in the TMV vector in order to produce them as soluble receptors, fused to tag peptide for facilitation of their purification and further immobilization.

Another advantage in using TMV as a vector is the fact that the heterologous sequence is driven by a subgenomic promoter. The heterologous protein behaves completely independent from the virus, and can therefore be directed into different cellular compartments without interfering with the replication and expression of recombinant viral RNA.

In one embodiment of the invention, in order to modify plant glycosylation towards a more mammalian pattern, plant specific complex glycosylation is prevented by eliminating endogenous N-Acetylglucosaminyl transferase I (GnT I) activity. Downregulation/knocking out the GnT I gene is done by making transgenic plants that express the GnT I gene in sense or antisense orientation; these plants are analysed for their deficiency to add β1,2 xylose and α1,3 fucose.

Plants with no or little fucosyl or xylosyltransferase activity are used to express bFSH and its receptor using for example the TMV based expression vector. Xylosyltransferase and fucosyltransferase can be knocked out and at least one of several mammalian glycosyltransferases have to be expressed. Providing the xylosyltransferase and fucosyltransferase knock-outs and thereby reducing the unwanted glycosylation potential of plants is a feasible option because for example an Arabidopsis thaliana mutant mutated in the gene encoding N-acetylglucosaminyltransferase I was completely viable (Von Schaewen et al., 1993). As N-acetylglucosaminyltransferase I is the enzyme initiating the formation of complex glycans (Schachter, 1991), this plant completely lacks the xylose and fucose containing complex glycans.

In another embodiment, the invention provides a plant comprising a functional mammalian enzyme providing N-glycan biosynthesis that is normally not present in plants additionally comprising at least a second mammalian protein or functional fragment thereof that is normally not present in plants. It is provided by the invention to produce in plants a desired gonadotrophin or gonadotrophin-receptor having a mammalian-type of glycosylation pattern, at least in that said glycoprotein is galactosylated.

In a preferred embodiment, the invention provides a plant according to the invention wherein said functional mammalian enzyme providing N-glycan biosynthesis that is normally not present in plants comprises mammalian, such as human or bovine β1,4-galactosyltransferase. An important mammalian enzyme that is missing in plants is this β1,4-galactosyltransferase. cDNA's encoding this enzyme have been cloned from several mammalian species (Masri et al., 1988; Schaper et al., 1986). The enzyme transfers galactose from the activated sugar donor UDP-Gal in β1,4 linkage towards GlcNAc residues in N-linked and other glycans (FIG. 1). These galactose residues have been shown to play an important role in the functionality of antibodies (Boyd et al., 1995). β1,4galactosyltransferase has recently been introduced in insect cell cultures (Hollister et al., 1998; Jarvis and Finn, 1996) to extend the N-glycosylation pathway of Sf9 insect cells in cell culture, allowing infection of these cultures with a baculovirus expression vector comprising a nucleic acid encoding a heterologous protein. It was shown that the heterologous protein N-linked glycans were to some extent more extensively processed, allowing the production of galactosylated recombinant glycoproteins in said insect cell cultures. Also the introduction of the enzyme into a tobacco cell suspension culture resulted in the production of galactosylated N-liked glycans (Palacpac et al., 1999) of endogenous proteins. However, no heterologous glycoproteins were produced in these plant cell cultures, let alone that such heterologous proteins would indeed be galactosylated in cell culture. Furthermore, up to date no transgenic plants comprising mammalian glycosylation patterns have been disclosed in the art. Many glycosylation mutants exist in mammalian cell lines Stanley and loffe, 1995; Stanley et al., 1996). However, similar mutations in complete organisms cause more or less serious malfunctioning of this organism (Asano et al., 1997; Herman and Hovitz, 1999; Loffe and Stanley, 1994). It is therefor in general even expected that β1,4-galactosyltransferase expression in a larger whole than cells alone (such as in a cohesive tissue or total organism) will also lead to such malfunctioning, for example during embryogenesis and/or organogenesis. Indeed, no reports have been made until now wherein a fully grown non-mammalian organism, such as an insect or a plant, is disclosed having the capacity to extend an N-linked glycan, at least not by the addition of a galactose.

Surprisingly, the invention provides such a non-mammalian organism, in particular a plant having been provided with a functional mammalian enzyme providing N-glycan biosynthesis that is normally not present in plants, thereby for example providing the capacity to extend an N-linked glycan by the addition of a galactose. In this set of plants, formation of mammalian type of complex glycans is promoted by introducing mammalian glycosyltransferases. First, a 1,4-galactosyltransferase gene (β1,4 GT) is introduced in order to attach galactose to the Man3 (GlcNAc) 2 core. In a preferred embodiment, the α2,6-sialyltransferase (α2,6 ST) gene is introduced to the α1,4 GT plants to endow the N-glycans with terminal sialic acid residues. This is done by crossing transgenic α1,4-GT plants with transgenic α2,6 ST plants. The resulting plants function as hosts to produce recombinant gonadotrophin with terminal sialic acid and improved metabolic half life.

In a preferred embodiment, the invention provides such a plant wherein said enzyme shows stable expression. It is even provided that beyond said second mammalian protein a third mammalian protein is expressed by a plant as provided by the invention. The experimental part provides such a plant that comprises a nucleic acid encoding both an antibody light and heavy chain or fragment. Of course, it is not necessary that a full protein is expressed, the invention also provides a plant according to the invention expressing only a fragment, preferably a functional fragment of said second mammalian glycoprotein, such as a gonadotrophin or gonadotrophin-receptor, said fragment being characterised by for example a truncated polypeptide chain, or a not fully extended glycan, for example only extended with galactose.

In this invention functional fragments are understood to have at least one function in common with the original molecule. The activity should be of the same kind, not necessarily the same amount.

In a preferred embodiment, the invention provides a plant according to the invention wherein said second mammalian protein or functional fragment thereof comprises an extended N-linked glycan that is devoid of xylose and/or of fucose. As can be seen from for example FIG. 3, plant-derived galactosylated glycoproteins in general contain less xylose and/or fucose residues, as is for example demonstrated by the overwhelming detection by Western blot of galactose-bearing proteins of various molecular weights, whereas in the Western blot at corresponding molecular weight positions little or no xylose and/or fucose bearing proteins are detected. Furthermore, in plants comprising galactosylated glycoproteins quantitatively less xylose and/or fucose is detected than in the corresponding wild-type plants. If one would desire to further separate glycoproteins such as gonadotrophin or gonadotrophin-receptor comprising extended N-linked glycan that is devoid of xylose and/or of fucose, or to produce these in a more purified way, several possibilities are open. For one, several types of separation techniques exist, such as (immuno)affinity purification or size-exclusion chromatography or elecrtrophoresis, to mediate the required purification. Furthermore, another option is to use as starting material plants wherein the genes responsible for xylose and/or fucose addition are knocked-out.

In the detailed description the invention provides a plant according to the invention, in particular a tobacco plant, or at least a plant related to the genus Nicotiana. However, use for the invention of other relatively easy transformable plants, such as Arabidopsis thaliana, or Zea mays, or plants related thereto, is also provided.

Herewith, the invention provides a method for providing a transgenic plant, such as transgenic Nicotiana, Arabidopsis thaliana, or Zea mays, or plants related thereto, which are capable of expressing a recombinant protein, with the additional desired capacity to extend an N-linked glycan with galactose comprising crossing said transgenic plant with a plant according to the invention comprising a functional mammalian enzyme providing N-glycan biosynthesis that is normally not present in plants, harvesting progeny from said crossing and selecting a desired progeny plant expressing said recombinant protein such as gonadotrophin or gonadotrophin-receptor and expressing a functional mammalian enzyme involved in mammalian N-glycan biosynthesis that is normally not present in plants. In a preferred embodiment, the invention provides a method according to the invention further comprising selecting a desired progeny plant expressing said recombinant protein comprising an extended N-linked glycan et least comprising galactose. In the detailed description a further description of a method according to the invention is given using tobacco plants and crossings thereof as an example.

With said method as provided by the invention, the invention also provides a plant expressing said recombinant protein and expressing a functional mammalian enzyme involved in mammalian N-glycan biosynthesis that is normally not present in plants. Now that such a plant is provided, the invention also provides use of a transgenic plant to produce a desired glycoprotein or functional fragment thereof, such as gonadotrophin or gonadotrophin-receptor, in particular wherein said glycoprotein or functional fragment thereof comprises an extended N-linked glycan et least comprising galactose.

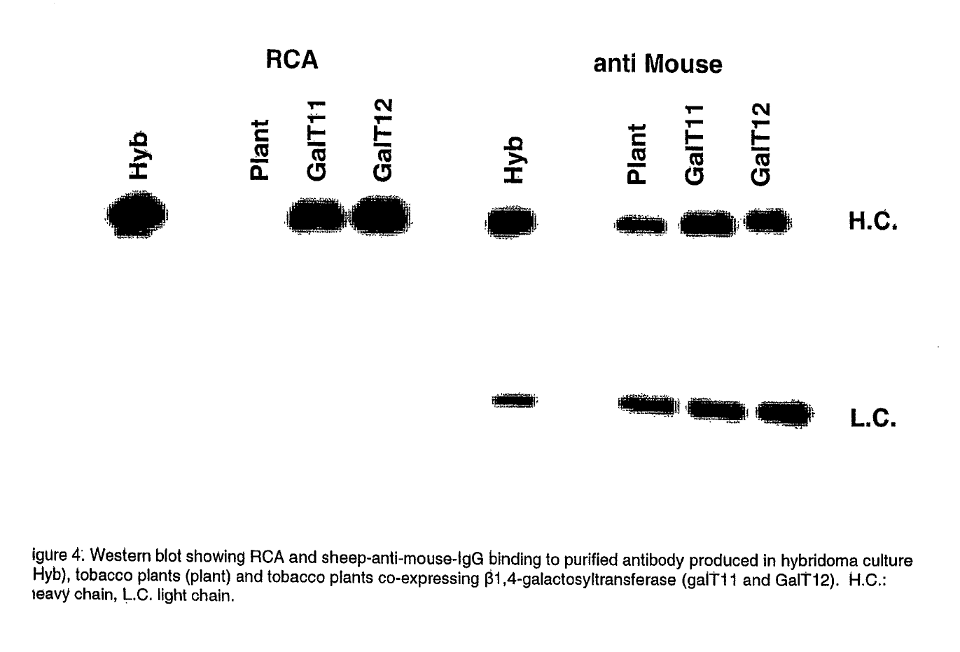

The invention additionally provides a method for obtaining a desired gonadotrophin or gonadotrophin-receptor or functional fragment thereof comprising for example an extended N-linked glycan at least comprising galactose; comprising cultivating a plant according to the invention until said plant has reached a harvestable stage, for example when sufficient biomass has grown to allow profitable harvesting, followed by harvesting said plant with established techniques known in the art and fractionating said plant with established techniques known in the art to obtain fractionated plant material and at least partly isolating said glycoprotein from said fractionated plant material. In the detailed description (see for example FIG. 4) an antibody having been provided with an extended N-linked glycan at least comprising galactose is provided.

The invention thus provides a plant-derived gonadotrophin or gonadotrophin-receptor or functional fragment thereof comprising an extended N-linked glycan at least comprising galactose, for example obtained by a method as explained above. The invention furthermore provides a plant-derived and at least partly purified gonadotrophin, such as bovine FSH, and its corresponding receptor, from transgenic plants infected with a recombinant virus such as modified TMV. Immuno-affinity chromatography combined with Centricon filtration has been shown to be an effective purification method in the case of insect cell-derived recombinant bFSH. Similar methods are developed for plant-derived rbFSH. Gonadotrophin-receptor is purified either by affinity chromatography (with insolubilized ligand or specific antisera) or by electro-elution after polyacrylamide gel electrophoresis. If productions are very abundant, classical separation techniques (size, hydrophobicity, etc.) can be used. Homologous transfected cell lines expressing receptors for bFSH are developed for in vitro measurement of bioactive recombinant bovine FSH. Several other types of assays are available already for monitoring of rbFSH during production and purification. For monitoring of gonadotrophin-receptor production and purification, assays have already been developed [immuno radio metric assay (IRMA), Western blotting] with respect to receptors of the porcine species. Likewise, these assays can be used for the measurement of bovine receptors.

Herewith, the invention also provides use of such a plant-derived gonadotrophin or gonadotrophin-receptor or functional fragment thereof according to the invention for the production of a pharmaceutical composition, for example for the treatment of a patient with an reproductive disorder. Such a pharmaceutical composition comprising a plant-derived gonadotrophin or gonadotrophin-receptor or functional fragment thereof is now also provided.

The invention furthermore provides plant-derived recombinant gonadotrophin such as bFSH and its receptor as very pure, stable and specific reagents in an assay method of commercial significance and as a therapeutic tool for assisted reproduction. The availability of plant-derived bovine gonadotrophins (FSH and LH), and of diagnostic testkits for the measurement of these substances provides a means of overcoming the limitations on assisted reproduction in for example cattle, as imposed by impure agents and lack of diagnostic tools. In animal reproduction, FSH is employed for increased production of eggs in cattle and for treatment of infertility in cattle and pigs.

In addition, the invention provides a plant comprising a cell comprising a functional mammalian enzyme or functional fragment thereof providing N-glycan biosynthesis additionally having been provided with an expression vector comprising a nucleic acid encoding a thyroid-stimulating hormone (TSH) or functional fragment thereof. TSH is another member (like FSH) of the glycoprotein hormone family (Grossmann et al, 1997, Endocrine Review, p 476-500) and essentially has the alpha subunit in common with FSH, which makes the FSH methods and plants provided herein of course easily applicable to the TSH field, if desired in combination with skills available in the art in said field.

Such a rbTSH plant as provided herein is preferably equipped with the enzyme human β1,4-galactosyltransferase, allowing the production of thyroid-stimulating hormone or functional fragment thereof that comprises an extended N-linked glycan, preferably galactose.

In another embodiment as provided herein, such rbTSH plant is essentially devoid of xylose and or fucose. Such a plant is obtained quit along the lines for rbFSH plants, whereby said expression vector is derived from a plant virus, for example a tobamovirus such as tobacco mosaic virus, which is most easily used with a tobacco plant. Of course, use of a rbTSH plant to produce a desired thyroid-stimulating hormone or functional fragment thereof, which preferably comprises an extended N-linked glycan et least comprising galactose, is also provided.

Furthermore, the invention provides a method for obtaining a thyroid-stimulating hormone or functional fragment thereof comprising cultivating a rbTSH plant as provided herein until said plant has reached a harvestable stage, harvesting and fractionating said plant to obtain fractionated plant material and at least partly isolating said thyroid-stimulating hormone or fragment thereof from said fractionated plant material. A plant-derived thyroid-stimulating hormone or functional fragment thereof comprising an extended N-linked glycan at least comprising galactose is hereby thus provided

Furthermore, the invention provides use of a recombinant thyroid-stimulating hormone or functional fragment thereof according to the invention for the production of a pharmaceutical composition. In particular such use according to the invention is provided for the production of a pharmaceutical composition for the treatment of a thyroid dysfunction and a pharmaceutical composition comprising a thyroid-stimulating hormone or functional fragment thereof according to the invention

The invention is further explained in the detailed description without limiting it thereto.

### DETAILED DESCRIPTION

One important enzyme involved in mammalian N-glycan biosynthesis that is not present in plants is β1,4-galactosyltransferase. Here, for one, the stable expression of β1,4-galactosyltransferase in tobacco plants is described. The physiology of these plants is not obviously changed by introducing β1,4-galactosyltransferase and the feature is inheritable. Crossings of a tobacco plant expressing β1,4-galactosyltransferase with a plant expressing the heavy and light chain of a mouse antibody produced antibody having terminal galactose in similar amounts as hybridoma produced antibodies. Herein it is thus shown that the foreign enzyme can be successfully introduced in plants. A clear increase in galactose containing glycoproteins is observed. Moreover, this feature is inheritable and there is no visible phenotypical difference between the galactosyltransferase plants and wild type. A mouse monoclonal antibody produced in these plants has a degree of terminal galactoses comparable to hybridoma produced antibody. This shows that not only endogenous proteins become galactosylated but also a recombinantly expressed mammalian protein.

### Materials and Methods

Plasmids and Plant Transformation

A plant transformation vector containing human β1,4-galactosyltransferase was constructed as follows: a 1.4 kb BamHI/XbaI fragment of pcDNAI-GalT (Aoki et al., 1992; Yamaguchi and Fukuda, 1995) was ligated in the corresponding sites of pUC19. Subsequently, this fragment was re-isolated using surrounding KpnI and HincII sites and cloned into the KpnI and SmaI site of pRAP33 (named pRAP33-HgalT). Using AscI and PacI sites the CaMV35S promotor-cDNA-Nos terminator cassette of pRAP33-HgalT was cloned in the binary vector pBINPLUS (van Engelen et al., 1995). Modifications to the published protocol are: After incubation with A. tum., leaf discs were incubated for three days in medium containing 1 mg/ml of NAA and 0.2 mg/ml BAP and the use of 0.25 mg/ml cefotaxime and vancomycine to inhibit bacterial growth in the callus and shoot inducing medium. 25 rooted shoots were transformed from in vitro medium to soil and, after several weeks, leaf material of these plants was analysed.

Northern Blotting

The β1,4-galactosyltransferase RNA level in the transgenic plants was analyzed by northern blotting (Sambrook et al., 1989) RNA was isolated from leafs of transgenic and control plants as described (De Vries et al., 1991). Ten μg of total RNA was used per sample. The blot was probed with a [³²P]dATP labeled SstI/XhoI fragment, containing the whole GalT cDNA, isolated from pBINPLUS-HgalT.

Glycoprotein Analysis

Total protein extracts of tobacco were prepared by grinding leafs in liquid nitrogen. Ground material was diluted 10 times in SDS page loading buffer (20 mM of This-HCl pH 6.8, 6% glycerol, 0.4% SDS, 20 mM DTT, 2.5 ig/ml Bromophenol Blue). After incubation at 100° C. for 5 min insoluble material was pelleted. Supernatants (12.5 μ/sample) were run on 10% SDS-PAGE and blotted to nitrocellulose. Blots were blocked overnight in 0.5% Tween-20 in TBS and incubated for 2 hours with peroxidase conjugated RCA₁₂₀ (Ricinus Communis Agglutinin, Sigma) (1 μg/ml) in TBS-0.1% tween-20. Blots were washed 4 times 10 minutes in TBS-0.1% tween-20 and incubated with Lumi-Light western blotting substrate (Roche) and analysed in a lumianalyst (Roche).

A rabbit polyclonal antibody directed against Horseradish peroxidase (HRP, Rockland Immunochemicals) was split in reactivity against the xylose and fucose of complex plant glycans by affinity chromatography with bee venom phospholipase according to (Faye et al., 1993). A rabbit anti LewisA antibody was prepared as described (Fitchette Laine et al., 1997). Blots were blocked with 2% milkpowder in TBS and incubated in the same buffer with anti-HRP, anti-xylose, anti-fucose or anti-Lewis-A. As secondary antibody alkaline HRP-conjugated sheep-anti-mouse was used and detection was as described above.

Plant Crossings

Mgr48 (Smant et al., 1997) is a mouse monoclonal IgG that has been expressed in Tobacco plants. The construct used for transformation was identical to monoclonal antibody 21C5 expressed in tobacco (van Engelen et al., 1994). Flowers of selected tobacco plants with high expression of β1,4-galactosyltransferase were pollinated with plants expressing Mgr48 antibody. The F1 generation was seeded and plants were screened for leaf expression of antibody by western blots probed HRP-conjugated sheep-anti-mouse and for galactosyltransferase expression by RCA as described above.

### Purification of IgG1 from Tobacco

Freshly harvested tobacco leaves were ground in liquid nitrogen. To 50 g of powdered plant material, 250 ml of PBS, containing 10 mM Na₂S₂O₅, 0.5 mM EDTA, 0.5 mM PMSF and 5 g polyvinylpolypyrrolid, was added. After soaking for 1 hour (rotating at 4° C.), insoluble material was removed by centrifugation (15 min, 15,000 g, 4° C.). The supernatant was incubated overnight (rotating at 4° C.) with 1 ml of proteinG-agarose beads. The beads were collected in a column and washed with 10 volumes of PBS. Bound protein was eluted with 0.1 M glycine pH 2.7 and immediately brought to neutral pH by mixing with 1 M Tris pH 9.0 (50 μl per ml of eluate).

Purified antibody was quantified by comparison of the binding of HRP-conjugated sheep-anti-mouse to the heavy chain on a western blot with Mgr48 of known concentration purified from hybridoma medium (Smant et al., 1997). Hybridoma Mgr48 and plant produced Mgr48 was run on 10% SDS-PAGE and blotted as described above. Detection with RCA was as described above. For antibody detection, blots were probed with HRP-conjugated sheep-anti-mouse and detected with Lumi-Light western blotting substrate as described above.

### Results

Human β1,4-galactosyltransferase Galactosylates Endogenous Proteins in Nicotiana tobacum

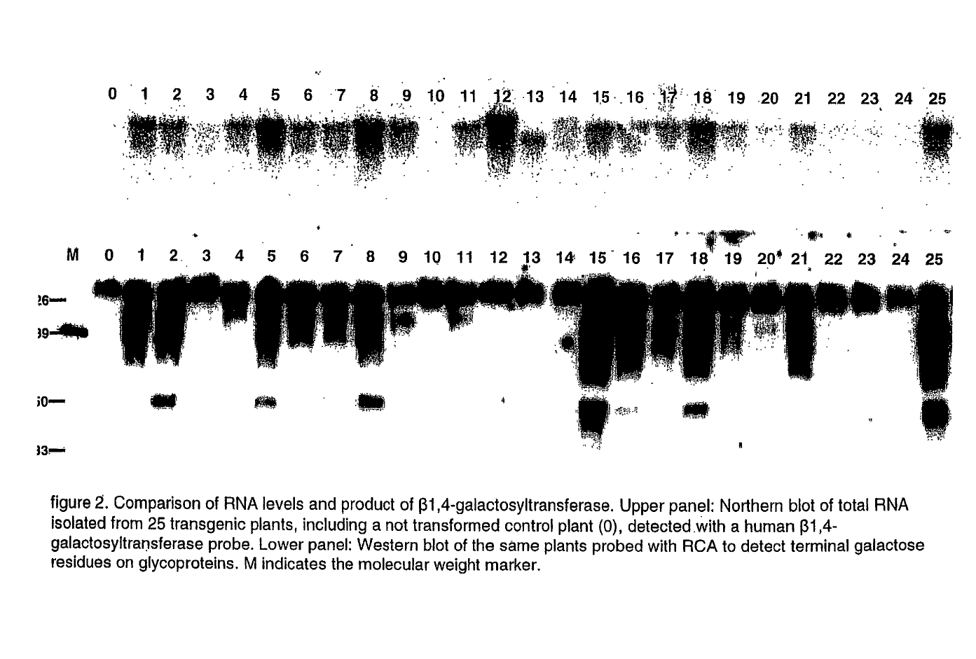

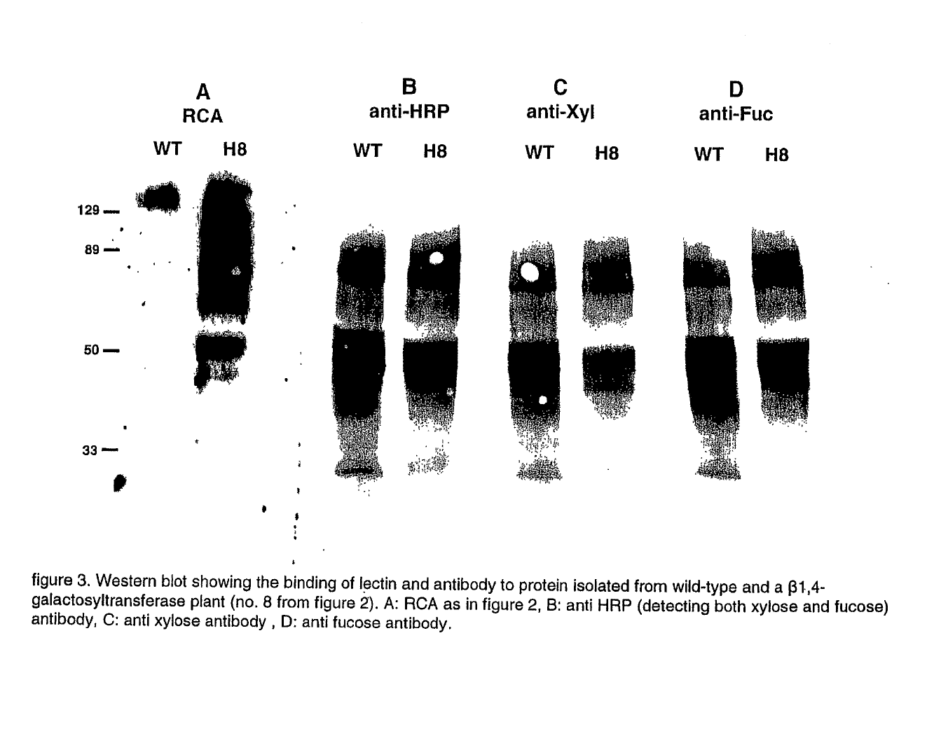

Human β1,4-galactosyltransferase (Masri et al., 1988) was introduced in tobacco plants by Agrobacterium mediated leaf disk transformation of plasmid pBINPLUS-HgalT containing a cDNA that includes a complete coding sequence. Twenty-five plants selected for kanamicin resistance were analysed for mRNA levels by northern hybridization (FIG. 2A). The same plants were analyzed by the galactose binding lectin RCA₁₂₀ (Ricinus Cummunis Agglutinin). RCA binds to the reaction product of β1,4-GalT (Galβ1,4GlcNAc) but also to other terminal β-linked galactose residues. RCA binds to one or more high molecular weight proteins isolated from non transgenic control tobacco plants (FIG. 2B). Probably these are Arabinogalactan or similar proteins. RCA is known to bind to Arabinogalactan proteins (Schindler et al., 1995). In a number of the plant transformed with Human β1,4-galactosyltransferase, in addition, binding of RCA to a smear of proteins is observed. This indicates that in these plants many proteins contain terminal β-linked galactose residues. There is a good correlation between the galactosyltransferase RNA expression level and the RCA reactivity of the trangenic plants. Human β1,4-galactosyltransferase expressed in transgenic plants is therefor able to galactosylate endogenous glycoproteins in tobacco plants.

As it is known that galactosylated N-glycans are poor acceptors for plant xylosyl- and fucosyltransferase (Johnson and Chrispeels, 1987), the influence of expression of β1,4-galactosyltransferase on the occurrence of the xylose and fucose epitope was investigated by specific antibodies. A polyclonal rabbit anti-HRP antibody that reacts with both the xylose and fucose epitope shows a clear difference in binding to isolated protein from both control and transgenic plants (FIG. 3).

Recombinantly Produced Antibody is Efficiently Galactosylated

The effect of expression of β1,4-galactosyltransferase on a recombinantly expressed protein was investigated. Three tobacco plants expressing β1,4-galactosyltransferase (no. GalT6, GalT8 and GalT15 from FIG. 2) were selected to cross with a tobacco plant expressing a mouse monoclonal antibody. This plant, expressing monoclonal mgr48 (Smant et al., 1997), was previously generated in our laboratory. Flowers of the three plants were pollinated with mgr48. Of the F1 generation 12 progeny plants of each crossing were analysed for the expression of both antibody and β1,4-galactosyltransferase by the method described in materials and methods. Of crossing GalT6xmgr48 and GalT15xmgr48 no plants were found with both mgr48 and GalT expression. Several were found in crossing GalT8xmgr48. Two of these plants (no. 11 and 12), were selected for further analysis.

Using proteinG affinity, antibody was isolated from tobacco plants expressing mgr48 and from the two selected plants expressing both mgr48 and β1,4-galactosyltransferase. Equal amounts of isolated antibody was run on a protein gel and blotted. The binding of sheep-anti-mouse-IgG and RCA to mgr48 from hybridoma cells, tobacco and crossings GalT8xmgr48-11 and 12 was compared (FIG. 4). Sheep-anti-mouse-IgG bound to both heavy and light chain of all four antibodies isolated. RCA, in contrast, bound to hybridoma and GalT plant produced antibody but not to the antibody produced in plants expressing only mgr48. When the binding of sheep-anti-mouse-IgG and RCA to the heavy chain of the antibody is quantified, the relative reaction of RCA (RCA binding/sheep-anti-mouse-IgG binding) to GalT8xmgr48-11 and 12 is respectively 1.27 and 1.63 times higher than the ratio of hybridoma produced antibody. This shows that RCA binding to the glycans of antibody produced in GalT plants is even higher than to hybridoma produced antibody. Although the galactosylation mgr48 from hybridoma is not quantified, this is a strong indication that the galactosylation of antibody produced in these plants is very efficient. There is a need for an accessible and standardised source of FSH for therapeutic and diagnostic purposes, which is guaranteed to be free of LH activity.

FSH preparations normally are derived from ovine or porcine pituitaries, which always implies the presence of (traces of) LH, and the risk of contamination with prion-like proteins. Substitution of brain derived FSH for plant produced recombinant FSH may be a good method of eliminating these problems. Furthermore application of plant produced FSH receptor (FSHR) in a diagnostic testkit provides a good method for measurement of bioactive FSH by receptor assay. However, production of bioactive animal glycoproteins in plants, especially for therapeutic purposes, requires modification of plant-specific sugar sidechains into a mammalian type of glycans. The invention provides recombinant bFSH and bFSHR by infecting stably transformed tobaccoplants capable of forming mammalian type of glycans, with recombinant Tobacco Mosaic Virus TMV containing the genes for bFSH or bFSHR.

Construction of Single Chain (sc) bFSH into pKS (+) Bluescript Vector, Construction of sc-bFSH-TMV and sc-bFSH-HIS-TMV

In order to circumvent the need of simultaneous expression of the two separate genes of bFSH-alpha and bFSH-beta subunits in plants, we decided to construct a bFSH fusion gene.

By overlap PCR we fused the carboxyl end of the beta subunit to the amino end of the alpha subunit (without a linker). In addition, we constructed a second sc-bFSH version carrying a 6× HIS tag at the C-terminus of the alpha subunit, which will allow us to purify the recombinant protein from the plant. Both, sc-bFSH and sc-bFSH-HIS constructs were subcloned into the cloning vector pKS(+) bluescript. The correctness of the clones was confirmed by sequence analysis.

Sc-bFSH was subcloned into the TMV vector. Two positive clones were chosen to make in vitro transcripts and Inoculate N. bentahamiana plants. After a few days, plants showed typical viral infection symptoms, which suggested the infective capacity of the recombinant TMV clones. In order to test whether the sc-bFSH RNA is stably expressed in systemically infected leaves, 8 days post inoculation RNA was isolated from infected N. benthamiana leaves and a reverse transcriptase polymerase chain reactions using bFSH specific primers was performed. In all cases we obtained a PCR fragment of the expected size, indicating the stability of our Sc-bFSH-TMV construct. Extracts of infected plants are used for Western blot analyses and ELISA to determine whether Sc-bFSH is expressed and folded properly.

Molecular Cloning of Full Length cDNA Encoding the Bovine FSH Receptor; Cloning of the Extracellular Domain of the FSH Receptor in TMV Vector.

Oligonucleotide primers based on partial published sequence date were designed for PCR amplification of nucleotide 1 to 1100 and 650 to 2150, respectively, from a bovine testicular cDNA library. The two fragments were subcloned in the pGEM-T vector (Stratagene), and fully sequenced. A unique common internal restriction site (XbaI) allowed the fragment ligation while subcloning into the eukaryotic expression vector pEE14. After plasmid amplification of a recombinant clone, transfection of CHO-K1 cells is the next step. Stable transfectants are usually obtained in three weeks. Functional experiments (hormone specific binding and transduction) will allow selection of the best expressing clones before the amplification process with increasing amounts of MSX in cell media.

In order to obtain a soluble FSH receptor, a fragment encoding part of its N-25 terminal extracellular domain was obtained by using PCR. The size of the soluble receptor (293 amino acids) has been chosen in order to retain all hormone interaction, and favor processing by elimination of the C-terminal Cystein cluster. Amplimers bearing appropriate restriction sites for subcloning the TMV vector were designed. After amplification and cloning, a synthetic DNA encoding the FLAG epitope (sequence=DYKDDDDK) as well as a stop codon was ligated. Subcloning of the construct into the TMV vector is now in progress.

In order to express the bovine FSH in a transient plant expression system the following constructs were cloned in a TMV expression vector:

the individual subunits of bFSH: α and β

a bFSH single chain (sc-bFSH) with the carboxy end of the β subunit fused to the amino end of the α subunits (according to Sugahara et al., 1996)

the single chain tagged with 6×HIS at the carboxy end (sc-bFSh-HIS)

After inoculation of Nicotiana benthamiana plants with in vitro transcripts from these constructs in all cases systemic infection of the recombinant viral constructs were obtained.

By reverse transcription-PCR analysis we could demonstrate the in planta stability of the hybrid TMV genomes carrying the bFSH sequences. Surprisingly also the co-transfected alpha and beta bFSH constructs were stably propagated in the same plants.

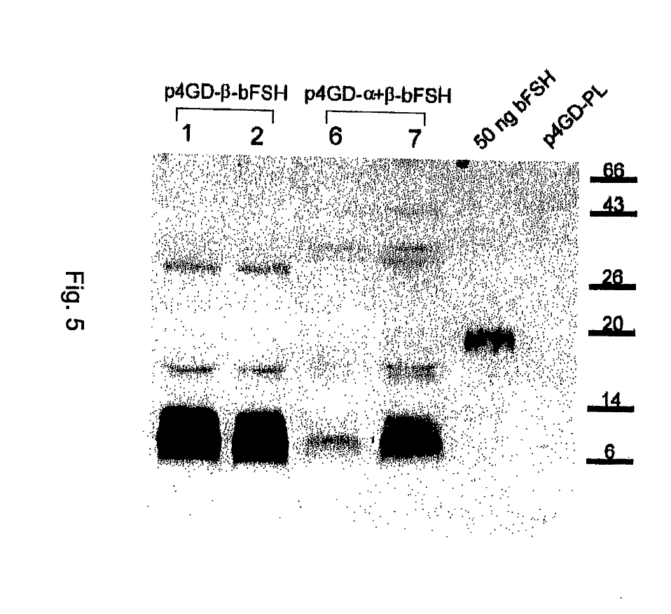

For detection of recombinant bFSH Western blot analysis of crude protein extracts from leaf material was carried out. Using an anti-human FSH beta subunit antiserum (FIG. 5) we could demonstrate the expression of the β bFSH (also in α/β cotransfected plants), as well as the expression of the sc-bFSH and the sc-bFSH-HIS. The beta-bFSH appeared as a double band at about 14 kDa. A major band at about 30 kDa was observed for the sc-bFSH and sc-bFSH-HIS. No signals were observed in TMV-infected extracts.

The presence of the 6×HIS tag on the sc-bFSH could be demonstrated using anti-HIS monoclonal antibodies. In a small scale protein miniprep using Ni-NTA agarose we were able to purify the sc-bFSH-HIS under denaturing conditions.

In order to investigate whether the sc-bFSH is glycosylated or not an enzymatic glycan digestion, PNGaseF digestion, was carried out. A clear band shift of bFSH treated with PNGaseF on Western blot analysis indicated the presence of N-glycans.

As sc-bFSH was detected almost exclusively in the soluble fraction after fractionating crude protein extracts with 100.000 × g, clearly the sc-bFSH is secreted by the plant cells into the extracellular space. Intercellular washing fluit (IF) extractions from leaf material were carried out. As shown by Western blot analysiss the sc-bFSH was clearly enriched in these IF fractions indicating a secretion of protein into the extracellular space.

Expression of Biologically Active Glycoforms of Bovine Follicle Stimulating Hormone in Plants

The follicle-stimulating hormone (FSH) is a pituitary glycoprotein hormone which regulates the ovarian follicle and testicular tubule development in all vertebrate species. In particular ovine, porcine or equine FSH are widely used to induce superovulation for human assisted reproduction of cattle and can benefit from homologous (i.e. bovine) recombinant FSH being free of potentially infectious material and other contaminating hormones. Here we describe the application of a plant based transient expression system for the rapid production of bFSH and its biochemical, immunological and biological. characterisation. We have used a tobacco mosaic virus-based vector to express bFSH in the tobacco related species Nicotiana benthamiana. The genes encoding the beta and alpha subunits were introduced in tandem into the viral vector to produce a single-chain bFSH (sc-bFSH) protein. N. benthamiana plants infected with recombinant viral RNA secreted high levels, up to 3% of total soluble proteins, of sc-bFSH to the extracellular compartment (EC). In-situ indirect immunofluorescence revealed consistently that the plant cell is capable of efficiently targeting the mammalian secretory protein to the extracellular destination. Mass spectrometry analysis of the N-glycans of immunoaffinity purified sc-bFSH derifed from EC fractions revealed two species of the plant paucimannosidic glycan type, derivatives of complex-type N-glycans. Crude nonpurified protein extracts from the EC were used for in vitro and in vivo bioactivity assays. The sc-bFSH exhibited bioactivity as it was able to induce cAMP production in CHO cell line expressing the porcine FSH receptor. Furthermore, in superovulatory treatments of mice, sc-bFSH displayed significant in vivo bioactivity, although comparably low with respect to pregnant mare serum gonadotrophins. We conclude that the rapid expression system used in this work may have a broad utility for the application of plant derived animal proteins in pharmaceutical products even for proteins where glycosylation is essential for function.

The follicle-stimulating hormone (FSH) is a pituitary glycoprotein hormone which regulates the ovarian follicle and testicular tubule development in all vertebrate species (Pierce and Parsons, 1981; Bielinska and Boime, 1995). Together with luteinizing hormone (LH), thyroid stimulating hormone (TSH) and chorionic gonadotrophin (CG), FSH forms the glycoprotein hormone family, which is the structurally most complex hormone family in the animal kingdom. These hormones are composed of two non covalently associated subunits, a common alpha subunit, and an unique beta subunit which confers biological specificity to each of these hormones. Each subunit forms intrachain disulfide bridges and carries two N-linked oligosaccharides, which is necessary for proper folding and secretion of the hormones (Suganuma et al., 1989, Feng et al., 1995). The N-linked carbohydrate chains of FSH exhibit considerable variation in both size and structure, including the degree of terminal sialyation and/or sulfation (Baenzinger and Green, 1988). The functional significance of this diversity of isoforms is not yet fully understood, but sialic acid seems to be the major determinant for the circulatory stability of FSH by preventing its rapid clearance mediated by the hepatic asialo-glycoprotein receptor (Drickamer, 1991). Besides this influence upon plasma halflife, the glycan heterogeneity is thought to provide a fine tuning mechanism with which to control gonadal function.

The technique of superovulation and embryo transfer is widely used to increase the number of offspring of genetically superior cows. Induction of superovulation is usually performed using pregnant mare serum gonadotrophin (PMSG) or pituitary derived FSH. Apart from containing potentially infectious viral or prion material, these preparations have the disadvantage of always containing some LH which is thought to contribute to the high variation in the results of treatments (Wilson J M et al., 1993).

To overcome these problems, an attempt was made to produce recombinant bovine FSH (rbFSH) in the baculovirus expression system (van de Wiel et al., 1998). Two main problems were associated with rbFSH: first, the glycans apparently did not contain terminal sialic acid, due to a probable complete inability of the insect cell line to perform sialyation (for a review see: Altmann et al., 1999). Second, although a relatively high yield of rbFSH was obtained in the baculovirus expression system, sufficient upscaling of production has not been achieved yet, especially because a decrease of expression rates in large scale fermentations using higher cell densities (Taticek et al., 1994).

In the recent years, the expression of recombinant proteins in plants has become a matter of interest. As an eucaryotic system, plant cells are capable of targeting recombinant proteins to the secretory pathway and of carrying out posttranslational modifications including disulfide bridge formation and glycosylation (Hiatt et al. 1989; Ma et al., 1995, Cabanes-Macheteau 1999). The N-glycosylation in higher organisms is conserved but differs in details. The processing of N-linked glycans occurs along the secretory pathway and complex-type N-glycans arise and are modified in the Golgi apparatus. Since some of the modifications are specific for an expression system, the structure of mature complex N-glycans differ to some extent in plants and mammals. In particular, plant glycoproteins do not bear sialic acid and carry a(1,3)-fucose and b(1,2)-xylose attached to their proximal N-acetylglucosamine which have not been found in mammals (for recent review see Lerouge et al., 1998). Since plants are gaining acceptance for the expression of recombinant therapeutic proteins (e.g. Ma et al., 1998 Tacket et al., 2000), it is important to examine in detail to what extent glycans of mammalian glycoproteins produced in plants differ from the original ones, and could influence their physiological properties. In this instance, glycoprotein hormones offer a particularly demanding model since proper N-glycosylation is required for folding, subunit assembly, intracellular trafficking and biological activity.

In this study, we used a plant viral vector to transiently express the bovine FSH in the tobacco related species Nicotiana benthamiana. This viral system uses a hybrid tobacco mosaic virus (TMV) to express foreign genes systemically in whole plants (Casper and Holt 1996). The levels of proteins expressed from TMV-based vectors are generally much higher than that obtained by stably transformed transgenic plants (Kusnadi et al., 1997, McCormick et al., 1999, Krebitz et al., 2000). Another advantage of this system is the speed of recombinant protein production, which is based on the rapid systemic movement of TMV in plants. Genes encoding the beta and alpha subunits were introduced in tandem into the viral vector to produce a single-chain bFSH (sc-bFSH) protein. Using different approaches, such as biochemical fractionation and in-situ indirect immunofluorescence, we were able to show that the mammalian protein is targeted to the secretory pathway and is efficiently secreted to the periplasmic space of the N. benthamiana cells. Using mass spectrometric a detailed N-glycan structure profile of the immunoaffinity-purified plant-produced hormone was obtained. Furthermore, using crude sc-bFSH extracts we demonstrated in vitro bioactivity, through receptor binding and activation of CHO cells, and in vivo bioactivity, through superovulation of fecundable oocytes in mice.

### Material and Methods

Construction of p4GD-sc-bFSH

In order to construct the single chain bFSH (sc-bFSH) with the carboxyl end of the b-subunit fused to the amino end of the a subunit (Sugahara et al., 1996) a gene SOEing strategy (Horton, 1993) was chosen (FIG. 1): The bFSH a subunit was amplified from the plasmid bovALPHA-pSP64 #1 (Leung et al., 1987) using the primers FSH-F 5′-GGA AAT CAA AGA ATT TCC TGA TGG AGA GTT TAC AAT GCA G-3′, containing 13 bp of the b subunit's carboxyl end, and Nsi-STOP 5′-AGC TAT GCA TCT ATT AGG ATT TGT GAT AAT AAC A-3′. The bFSH b subunit was amplified from the plasmid Bov FSHbeta pGEM3 (Maurer and Beck, 1986) using the primers FSH-A 5′-ATA TGA GTC GAC ATG AAG TCT GTC CAG TTC-3′ and FSH-E 5′-CTC CAT CAG GAA ATT CTT TGA TTT CCC TGA AGG AGC AGT A-3′, the latter including 13 bp of the a 5′-end. The resulting 2 fragments which contain a 26 bp overlapping region were combined in 5 PCR extension cycles with an annealing temperature of 45° C. Subsequently, this overlap PCR product was amplified by PCR using the primers FSH-A and Nsi-STOP. Following SalI/NsiI digestion, this fragment was ligated to SalI/PstI restricted TMV based expression vector p4GD-PL (Casper and Holt, 1996), resulting in the construct p4GD-sc-bFSH. This construct was used for all expression experiments.

Plant Material and Inoculation of Plants

Nicotiana benthamiana plants were grown in a controlled growth chamber with 22° C. day and night temperature, 50% humidity and 16 h light period. The recombinant viral vectors p4GD-sc-bFSH and p4GD-PL (as negative control) were linearized by SfiI digestion. Capped in vitro run off transcripts were made using a T7 transcription kit (RiboMax, Promega Ltd., Wis., U.S.A.). In vitro transcripts were used to mechanically inoculate N. benthamiana plants at a six leaf stage. Symptoms of infections were visible 8-10 days post inoculation (dpi) as leaf deformations, with some variable leaf mottling and growth retardation.

Reverse Transcriptase Polymerase Chain Reaction (RT-PCR) Analysis of Infected Plants

12-16 dpi the replicative stability of the hybrid TMV RNA-genome derived from p4GD-sc-bFSH was investigated. Total RNA from systemically infected leaves was prepared using TriReagent (Molecular Research Centre, Inc.) and cDNA synthesis (reverse transcription) was performed using the TMV (p4GD-PL) specific reverse primer p4GD-RV 5′-TTT TTC CCT TTT TTG TTT TCC G-3′ located downstream the multiple cloning site. Using p4GD-RV and the TMV specific forward primer p4GD-FW 5′-GAT GAT GAT TCG GAG GCT ACT-3′ which anneals upstream of the multiple cloning site, a specific RT-PCR fragment of 826 bp was expected for the sc-bFSH construct. As negative control the same procedure was also carried out on wt TMV (p4GD-PL) infected plants. In this case a RT-PCR product of 145 bp was expected.

Total Soluble Protein Extraction From sc-bFSH Expressing N. benthamiana Leaves

Two to three weeks after inoculation, systemically infected leaves were harvested, and total soluble protein (TO) was extracted by grinding the leaves in 10 vols (w/v) of 10 mM TRIS-HCl pH 7.6 buffer. Cellular debris were sedimented (15 min, 6000 rpm, 4° C.). The supernatant was used for SDS-PAGE. The same procedure was also carried out on leaves which were subjected to intercellular washing fluid extraction (see below).

Preparation of Intercellular Washing Fluid (IF) from sc-bFSH Expressing Leaves

In order to enrich periplasmic (extracellular) proteins an intercellular washing fluid (IF) extraction was carried out using the method described by Pogue et al. (1997) with modifications. Systemically infected leaves from sc-bFSH expressing plants, were harvested two to three weeks after inoculation. The tissue was rolled lengthwise in parafilm® (American National Can, Ill., U.S.A.), inserted into a 50 ml plastic tube and submerged in pre-cooled (4° C.) 10 mM TRIS-HCl pH 7.6 buffer. A vacuum was applied twice for 1 min, with a rapid release of the vacuum to infiltrate the leaves with buffer. Subsequently, the leaves were blotted dry to remove excess buffer, were again rolled in parafilm and inserted into a centrifuge tube. The IF was collected by a downspin at 3,000 × g for 15 min at 4° C. The IF was finally clarified by a centrifugation at 10.000 × g for 15 min at 4° C and the supernatant used for analyses. The same IF preparations were made from plants infected with p4GD-PL (wt TMV) or not infected wildtype plants serving as negative controls.

Fractionation of sc-bFSH Containing Plant Extracts by Ultracentrifugation

Crude total protein extracts (TO) from sc-bFSH expressing plants were prepared as described above, followed by ultracentrifugation at 100.000 × g for 1 h at 40° C. to obtain a pellet (P) and a supernatant (SN) fraction. The pellet was resuspended in 10 mM TRIS-HCl pH 7.6 in the same volume as the supernatant fraction. Equal volumes of total extract, pellet and supernatant fractions were subjected to Western blot analysis (see below).

N-Glycosidase F Digestion of sc-bFSH

The procedure was as described by Tretter et al., 1991 with modifications. IF extraction from sc-bFSH expressing plants (see above) was conducted using 50 mM TRIS-HCl, 20mM EDTA, pH 8.0, followed by an addition of b-mercaptoethanol and SDS to each 0.5% (v/v, w/v, respectively). 10 ml of this extract were heated at 95° C. for 5 min, followed by an addition of 40 ml 50 mM TRIS-HCl, 20 mM EDTA, pH 8.0 and 1% IGEPAL, (Sigma, Mich., U.S.A.). Finally, 1 ml of N-glycosidase F, peptide-N⁴-[N-acetyl-b-glucosaminyl] asparagine amidase, (20 mU/ml; Roche, CH) was added, followed by an incubation at 37° C. overnight (16 h). After digestion, the proteins were precipitated using aceton and subjected to Western blot analysis. As a control, the same procedure was done without the addition of N-glycosidase F.

Western Blot Analysis of sc-bFSH Extracts

Protein extracts prepared as described above were electrophoresed on 12.5% SDS-polyacrylamide gels under reducing conditions (Lammli, 1970). Following electroblotting onto nitrocellulose (Amersham Life Science Ltd, U.K.), the blots were blocked with 5% non-fat dry milk in TBS containing 0.1% Tween 20 (Sigma, Mich., U.S.A.). The primary antibody was an anti-human FSH b-subunit (R812, reference!) rabbit polyclonal antiserum diluted to 1:2500 in TBS containing 0.1% Tween (TTBS) and 1% BSA (Sigma, Mich., U.S.A.). As secondary antibody an anti-rabbit IgG goat polyclonal antiserum-horde radish peroxidase conjugate (Sigma, Mich., U.S.A.), diluted to 1:20000 in TTBS, was employed. Detection was done using an enhanced chemiluminescence substrate (ECL, Super Signal, Pierce, Ill., U.S.A.). For quantification of the signal intensities Kodak Digital Science 1D Image Analysis Software was used.

In parallel to the immunodetections, the total protein contents of the different extracts were visualised using silver staining of the gels (Amersham Pharmacia Biotech AB). Quantitation of total protein was done using a BCA protein assay kit (Pierce, Ill., U.S.A.).

Localisation of sc-bFSH by In Situ Indirect Immunofluorescence

Indirect in situ immunofluorescence was performed according to Goodbody et al. (1994) and Flanders et al. (1990) with modifications. All solutions were made in microtubule stabilizing buffer (MTSB): 50 mM PIPES, 5 mM EGTA, and 5 mM MgSO₄, pH 6.9. Epidermal tissue sections (leaf stalk) from sc-bFSH expressing plants, prepared as described above, were fixed in 4% (v/v) formaldehyde for 60 min, followed by four washing steps in MTSB over 60 min. In order to aid antibody penetration, the tissue was cross hatched with a flexible, double sided razor blade. Permeabilisation was performed by 10% DMSO and 0.4% IGEPAL (Sigma) for 15 min. An additional blocking step was performed by incubating in 1% BSA for 15 min. Both antibody incubations were carried out for 60 min followed by three washing steps with the blocking solutions after the primary incubation and with MTSB after the secondary incubation. As primary antibody the rabbit anti-hFSH-b-peptide polyclonal antibody (R812) was used, the secondary antibody was a Cy3 conjugated sheep anti-rabbit antibody (Sigma). As anti-fade mounting reagent, CITIFLUOR was used (City University, London).

Microscopic Imaging

Imaging was conducted on a Biorad MRC 600 confocal laser scanning microscope equipped with a Krypton/Argon mixed gas laser and a ×40 objective. Excitation of the Cy3 fluorochrome was done at 569 nm using the YHS filter block. Phase contrast illumination of the tissue sections was performed on the same sections. Images from the confocal system were imported into PaintShopPro 5.01 (Jasc Software, Inc., Minn., U.S.A.) for colorisation.

Immunoaffinity Chromatography

Immunoaffinity chromatography was essentially done as described previously (Van de Wiel et al., 1998) and according to the manufacturer's instructions. The gel pellet was washed 2 times with PBS13 and then incubated with 30 ml of sc-bFSH The purity and concentration of sc-bFSH in the eluate fractions were monitored on a silver stained SDS-PAGE-gel, on which the sc-bFSH appeared as a single band. For mass spectrometry analysis (see below), a volume of pure sc-bFSH corresponding to approximately 8 mg was first dialyzed against deionized water using Slide-a-lyzer® MINI dialysis units having a molecular weight cut off of 10 kDa (Pierce, Ill., U.S.A.) to remove salts and glycine. Subsequently, the dialyzed fractions were concentrated by lyophilisation, and dissolved in SDS-PAGE sample buffer.

N-glycan Analysis of sc-bFSH by Matrix Assisted Laser Desorption/Ionisation Mass Spectrometry (MALDI-MS)

The procedure was carried out as described by Kolarich and Altmann (in press) with modifications. In brief, approximately 8 mg of immunoaffinity-purified sc-bFSH were electrophoresed on a reducing 12.5% C 1%T SDS-polyacrylamide gel system. Following electrophoresis, the gel was stained using a silver staining method which is compatible with mass spectrometry analysis (Schevchenko et al. 1996). The band of interest was excised with a scalpel, and after washing, reduction and S-carboxamidomethylation subjected to tryptic in-gel digestion as described by Jensen et al. (1997). In order to identify the sc-bFSH, the extracted peptides were dissolved in 10 ml 5% (v/v) formic acid and analysed on a DYNAMO (ThermoBioAnalysis, Ltd.) linear time-of-flight MALDI-MS (peptide mapping). 0.2 ml of the sample was dried on the plates followed by addition of 0.8 ml matrix solution (1% (w/v) a-cyano-4- hydroxycinnamic acid in 70% (v/v) acetonitrile. Peptides were measured with a <<dynamic extraction >> setting 0.1. Average masses of [M+H]⁺ ions were determined using human bradykinin and human renin substrate tetradecapeptide for external calibration of the instrument. The ExPASy <<Peptide-Mass >> program was used to construct theoretical peptide maps of sc-bFSH.

Following peptide mapping, the N-glycans in the residual aliquot of the tryptic digest were released by peptide-N⁴-(N-acetyl-b-glucosaminyl) asparagine amidase A (N-glycosidase A, Roche, CH). To remove peptides and salts, the digest was loaded onto a triphasic microcolumn consisting of anion exchange, reversed phase and a mixture of polyamide/cation exchange resins. For MALDI-MS analysis, the samples were redissolved in 10 ml water. 1 ml of the sample was spotted onto the target, dried under vacuum, followed by addition of 0.8 ml matrix solution (a 1:1:1 mixture of 2% (w/v) 2,5-dihydroxybenzoic acid in 30% (v/v) acetonitrile, 1% (v/v) D-arabinosazone in acetonitrile and 0.2 M 2.5-dihydroxybenzoic acid/0.06 M 1-hydroxy-isoquinoline in 50% (v/v) acetonitrile (DARCI). The oligosaccharides were analyzed with a <<dynamic extraction >> of 0.1. Compilation of the spectra was done manually by the addition of single shots. Average masses of [M+Na]⁺ ions were recorded using a partial dextran hydrolysate for external calibration of the instrument.

In Vitro Bioactivity Assay in FSH Receptor Expressing CHO Cells

IF-extracts from N. benthamiana plants infected by p4GD-sc-bFSH or p4GD-PL (negative control) were diluted in GMEM-S medium without calf serum in a final volume of 0.2 ml as indicated in FIG. 8, A. These extract dilutions were incubated on CHO cell layers expressing the porcine FSH receptor (Abdennebi et al., 1999) for 1 h 30 at 37° C. Known concentrations of pituitary bFSH were applied to cells in the same conditions (see FIG. 8, B). cAMP levels in supernatants were determined using a specific RIA (NEN-Dupont de Nemours, Les Ulis, France). All assays were performed in duplicate and repeated twice.

Superovulatory Treatment of Mice

15 6-8 week old female C57/CBA mice were treated each with 100 ml of 11 times concentrated sc-bFSH-IF extract (concentration was done using an AMICON ultrafiltration cell, MWCO 10 kDa). Serving as a negative control, 15 mice were treated each with 100 ml of 11 times concentrated IF-extract from not infected plants (NI-IF). Furthermore, the response of 14 mice to 5 IU pregnant mare serum gonadotrophin (PMSG, Folligon, Intervet) in the same volume was investigated. 46-48 hours post FSH or wt-IF injections, the 3 groups were treated with 5 IU human chorionic gonadotrophin (hCG; Chorulon, Intervet). To recover mature oocytes, superovulated females were sacrified 15 hours post-hCG-injection. Oocytes were incubated after collection in 0.5% hyaluronidase (Sigma) in PBS for 1-2 min at 37° C. to remove cumulus. The total number of oocytes were counted.

### Results

Vector Construction and Expression of sc-bFSH in N. benthamiana Plants

Although native bFSH is expressed from two different genes on different loci, we chose to genetically fuse the carboxyl end of the bFSH b subunit to the amino end of the a subunit according to Sugahara et al. (1996) in order to produce a single-chain bFSH (sc-bFSH). Both the receptor binding affinity and the potency of adenylate cyclase activation for the single-chain human FSH were shown to be similar to that of recombinant human FSH heterodimer (Sugahara et al. 1996). The fusion gene was inserted into the tobacco mosaic viral-based vector p4GD-PL (Casper and Holt 1996). In vitro run off transcripts of the construct p4GD-sc-bFSH were capable of infecting N. benthamiana plants systemically as indicated by clear mottling and mosaic symptoms on systemic infected leaves 10-14 days post inoculation (dpi). To determine the in planta replicative stability of the hybrid TMV RNA, reverse transcription PCR (RT-PCR) was carried out with RNA isolated from newly developed leaves 14 dpi. The amplification of a single RT-PCR fragment of expected size confirmed the presence of the FSH sequence in systemically infected TMV leaves. Further RT-PCR analyses were carried out until 28 dpi, in which likewise no instabilities of the p4GD-sc-bFSH derived viral RNA were observed.

17-21 dpi infected leaves were harvested, total soluble protein (TO) extracted and subsequently subjected to SDS-PAGE. Silver staining revealed the abundant coat protein at position 17 kDa and a diffuse additional band at position 30 kDa, the expected size of undissociated alpha-beta bFSH heterodimer (Wu et al., 1992), which was not present in extracts of control plants. The corresponding Western blot analysis using an anti-humanFSH beta (hFSHβ) antiserum clearly confirmed the expression of the hormone by displaying a strong signal at postition 30 kDa. Minor signals were obtained at 60 kDa which we interpret as artefactual dimerisation product that can occur during SDS-PAGE (Shi and Jackowski, 1998). Additionally, a putative degradation product of sc-bFSH was detected at approximately 15 kDa. It was not possible to reduce the amount of this degradation product by including a cocktail of plant protease inhibitors to the extraction buffer. The specificity of the signals obtained in sc-bFSH expressing plants was indicated by the absence of any signal in extracts from control plants.

Subcellular Localisation of sc-bFSH

In a series of experiments using different approaches we wanted to investigate whether the sc-bFSH as a glycoprotein hormone—in homology to the situation in mammals—is targeted to the secretory pathway of plant cells and if the protein is secreted into the periplasmic (extracellular) space.

Ultracentrifugation of total protein extracts (TO) from sc-bFSH expressing plants was carried out which resulted in a pellet (P) and a supernatant (SN) fraction. All three fractions, TO, P and SN, were subsequently analysed by SDS-PAGE. The different staining patterns of SN and P on a silver-stained gel indicated a selective enrichment of soluble and of mostly membrane associated proteins, respectively. Silver staining and immunoblot analyses using anti-hFSHb antibodies clearly revealed the enrichment of the hormone in the soluble SN fraction, which is consistent with a periplasmic location.

The fact that no hormone was detected in the P fraction excludes the formation of inclusion bodies which often is a consequence of protein overexpression.

As a next step a so-called intercellular washing fluid (IF), which is characterised by the specific enrichment of periplasmic (extracellular) proteins, was separated from the total protein extracts (TO) and compared with the remainder (RE) thereof. TO, IF and RE fractions were subjected to SDS-PAGE and silver staining revealed an additional diffuse band in the IF at the expected size of the hormone (30 kDa), which is absent in control IF fraction (FIG. 3). Immunoblotting clearly demonstrated the enrichment of the sc-bFSH in the IF fraction. We calculated an enrichment factor of 6-10 for sc-bFSH in the IF with respect to the TO fraction. Furthermore, the computer-assisted comparison of the signal intensities of 50 ng pit-bFSH with that of sc-bFSH in TO and IF fractions allowed us to estimate sc-bFSH concentrations of 0.4% and 3% of total soluble protein, respectively.

To confirm the periplasmic location of sc-bFSH, in situ indirect immunofluorescence was performed. Mechanical sectioning of epidermal cells of sc-bFSH expressing plants was used to provide entry sites for the antibodies. Hence, only cut cells show an immunostaining. The fluorescence signal was obtained in the periphery of the cells, being clearly different from a cytoplasmic or vesicular fluorescence staining as shown previously (Boevink et al., 1998; Essl et al., 1999). The in situ indirect immunostaining of sc-bFSH appeared as a thin film located inside of the cell wall being consistent with a periplasmic location.

N-glycosylation Analysis of sc-bFSH

Since native bFSH is a glycoprotein hormone and its N-glycosylation is essential for bioactivity, the N- glycosylation status of sc-bFSH was investigated. Sc-bFSH has four potential N-glycosylation sites and, in Western blot analyses, a diffuse band was detected at position 30 kDa, which is larger than the expected size of the unglycosylated (23 kDa) protein (FIGS. 2, 3). This already indicated the glycosylated status of the recombinant protein. As a first approach a glycan specific enzyme (PNGase F) digestion of IF extracts was made. PNGase F digests all oligosaccharide species except those containing the plant specific core α1,3 fucose (Tretter et al., 1991). Clearly the band detected at position 30 kDa shifted to a band of smaller size (26 kDa) indicating sensitivity of sc-bFSH to N-glycosidase F. This result demonstrated the presence of N-linked glycans lacking core α1,3 fucose. In addition, presence of a minor “smear” signal at position 26-27 kDa which is not susceptible to N-glycosidase F digestion, indicates the presence of a fraction carrying core α1,3 fucose residues.

The detailed structure of N-glycans attached to sc-bFSH was elucidated by MALDI-MS. This procedure was specially designed for the analysis of N-glycans potentially containing core fucose in a1,3 linkage. Immunoaffinity-chromatography using a monoclonal antibody against human FSH was carried out to purify the sc-bFSH from IF extracts, resulting in <<single band purity>> as evidenced by SDS-PAGE/silver staining. The sc-bFSH was subjected to tryptic in-gel digestion in order to provide susceptibility to the subsequent N-glycosidase A digestion. This further allowed the identification of the tryptic peptides measured by MALDI-MS (data not shown).

Subsequently, the enzymatically released N-glycans were cleaned up for MALDI-MS. The resulting mass revealed two peak masses that could be assigned to 2 known plant N-glycans of the paucimannosidic type: MMX and MMXF³. An analysis of the respective peak areas revealed a 4:1 ratio between MMX and MMXF³. Consistent with the result of the N-glycosidase F digestion, the mass spectrometric analysis revealed a glycan species, MMX, which is susceptible to N-glycosidase F digestion, and a second minor fraction, MMXF³, which is not.

FSH Receptor Activation by Plant sc-bFSH

To determine the in vitro bioactivity, the plant-expressed sc-bFSH was tested for its ability to induce cyclic AMP (cAMP) production in a CHO cell line expressing the porcine FSH receptor (pFSHR). Evidently, cAMP levels, as determined by RIA, were raised in a dose dependent manner upon addition of increasing amounts of pit-FSH. The effect of the plant expressed sc-bFSH on the production of cAMP in this cell line was determined by applying several nonpurified sc-bFSH-IF dilutions. Increasing concentrations of the sc-bFSH containing IF extract resulted in a dose responding cAMP production. The specificity of the cAMP production upon addition of plant-produced sc-bFSH was demonstrated by an absence of a response of the cells to an IF extract from control plants. The pit-bFSH standard curve allowed to estimate a concentration of 5 ng in vitro bioactive sc-bFSH per ml IF.

### Example of an Vivo Bioassay

Superovulatory Treatment of Mice

15 6-8 week old female C57/CBA mice were treated each with 100 ml of 11 times concentrated sc-bFSH-IF extract (concentration was done using an AMICON ultrafiltration cell, MWCO 10 kDa). Serving as a negative control, 15 mice were treated each with 100 ml of 11 times concentrated IF-extract from not infected plants (NI-IF). Furthermore, the response of 14 mice to 5 IU pregnant mare serum gonadotrophin (PMSG, Folligon, Intervet) in the same volume was investigated. 46-48 hours post FSH or wt-IF injections, the 3 groups were treated with 5 IU human chorionic gonadotrophin (hCG; Chorulon, Intervet). To recover mature oocytes, superovulated females were sacrified 15 hours post-hCG-injection. Oocytes were incubated after collection in 0.5% hyaluronidase (Sigma) in PBS for 1-2 min at 37° C. to remove cumulus. The total number of oocytes were counted. The total number of oocytes indicated a high superovulatory response of the mice to PMSG, where as much as approximately 4 fold more oocytes were counted as compared to the negative control. A significant, albeit comparably low, superovulatory response to sc-bFSH (1,5 fold above the negative control) was found. The mean number of oocytes for each group, WT-IF, sc-bFSH-IF and PMSG, and the respective standard deviations are illustrated.

Embryo Isolation and Culture

Female mice were treated with intraperitoneal injection of pregnant mare serum gonadotrophin (Folligon-intervet, 5 IU), sc-bFSH-IF extracts, WT-IF extracts, followed by human chorionic gonadotrophin (chorulan, intervet) 48 h later. As a control, untreated females that showed an oestrus behaviour were included in this study. Mice were caged overnight with males and 1 cell stage embryos were isolated 19-26 hours after hCG from females showing sperm vaginal plugs (day 1). The ampullary regions of excised oviducts was placed at 30° C. in PBS medium containing bovine serum albumine at 4 mg/ml together with bovine hyaluronidase (sigma) at 50 units/ml. After 3-5 minutes the cumulus cells were dissociated and the eggs washed several times in PBS medium. Fertilized eggs showing two pronuclei and polar body were pooled from several females of the same group. Embryos were cultured under paraffin oil (DBH) in 10 μl drops of Whitten's medium in an atmosphere of 5% CO2 in air at 37° C.

Embryos were cultured for up to 72 hours in vitro in Whitten's medium and examined several times. For each group, development was assessed by the proportion of fused eggs that became blastocysts. The results clearly indicated that, whatever the treatment, more than 80% of the fused eggs reached the morula stage 3, 5 days after fecondation while at 7, 5 days more than 50% of them developed into bastocysts. Our results clearly showed no differences between PMSG and plant FSH extract treated females. These experiments strongly suggested that crude extract of infected plants containing FSH did not induce any deleterious effects on mice embryo further development, indicating potential use for assisted reproduction.

### Example of Assays for Detecting the Presence of sc-bFSH-FSH Receptor Complexe

Two different assays were performed to detect the presence of sc-bFSH-FSH receptor complexe (FSHC) in tobacco leaves.

1. Elisa

The wells of M96 microtiterplate were coated with polyclonal antiserum against FSH receptor (dilution at 1/200). To each coated well, 100 μl was added of serial dilutions of either WT-IF extract or intracellular fluid from infected tobacco leaves containing FSHC.

After incubation (1 h at 37° C.) and washing, a monoclonal antibody against human FSHβ (0.01 mg/ml) was added and incubated (1 h at 37° C.) which was followed by washing and addition of anti-mouse IgG coupled to peroxidase (1:500, 100 μl/well)

After washing, TMB and H₂O₂ were added (for color development). The reaction was stopped by adding H₂SO₄.

2. Immunoradiometric Assay (IRMA)

The assay used coated beads for the capture of FSHC from infected tobacco leaves. Polystyrene beads were incubated overnight at 4° C. in the presence of polyclonal antibody against FSH receptor. After washing with distillated water, beads were used to capture antibody-reactive molecules present in the IF extract (WT or FSHC). After 2 h incubation at followed by extensive washing, labeled diluted monoclonal antibody against FSHβ (in 0.01 M PiNacl containing 50% Foetal Calf Serum) was added. The reaction mixture was incubated for 1 h at 20° C, followed by a washing step and residual radioactivity was counted.

Here we demonstrated the rapid and high level expression of a single-chain version of the bovine follicle stimulating hormone (sc-bFSH) in Nicotiana benthamiana plants using a tobacco mosaic virus based transient expression system. A combination of molecular and cell biological experimental approaches showed consistently that the plant cell is fully capable of directing a mammalian secretory protein such as a glycoprotein hormone to the extracellular destination. Hence, the native leader sequence of the beta subunit of bFSH (and accordingly also the bTSH subunit) which represents the N-terminus of the sc-bFSH is recognised by the plant cell and subsequently the protein is directed to the secretory pathway. This observation is in agreement with the correct recognition of mammalian signal peptides derived from antibodies by the plant cell machinery. Furthermore, correct formation of disulfide bridges and folding of the tethered hormone subunits similarly to its native counterpart pituitary bFSH was evidenced by the in vitro bioactivity assay.

Most clinically important mammalian proteins, such as TSH, FSH and LH, have N-glycans, which confer different biological functions, such as resistance to protease attacks, antigenicity, immunogenicity and, as for FSH, plasma clearance rates. Although N-glycosylation is conserved in higher organisms to some extent, so far no established heterologous expression system produces correct mammalian-type N-glycans, due to more or less differing biosynthetic pathways. The perspective to use plants as economic factories to produce therapeutic recombinant proteins at a low cost makes it important to investigate the capacity of plant cells to produce functional mammalian-like glycoproteins. Our detailed analysis on the N-glycosylation pattern of sc-bFSH constitutes a complete study of a mammalian glycoprotein. Surprisingly, only two oligosaccharide structures were found N-linked in sc-bFSH and were identified as paucimannosidic N-glycans containing b1,2 xylose and a1,3 fucose residues in a ratio 80:20%, respectively. Paucimannosidic glycans are considered as typical vacuole-type N-glycans, which result from the elimination of the terminal residues of complex-type N-glycans in post-Golgi compartments (for review see Lerouge et al. 1998). Secreted proteins in plants usually carry complex-type N-glycans with a high degree of heterogeneity (Melo et al., 1997, Ogawa 1996, for a review see Sturm 1995). Although paucimannosidic N-glycans have been found to a minor extent in secreted proteins, the presence of this type of glycan as predominant species is a rather unusual case. Still, to our knowledge, there is only one detailed comparative study of a mammalian glycoprotein, a mouse immunglobulin (“plantibody”), produced in a plant expression system (Cabanes-Macheteau et al., 1999). In contrast to sc-bFSH the plantibody shows a higher degree of N-glycan heterogeneity, as a total of 8 different species of oligosaccharides were found. However, since no detailed analysis of the protein location was done it cannot be excluded that fractions of plantibodies are stored in different compartments of the plant cell (Cabanes-Macheteau et al., 1999). To our knowledge this is the first report of a detailed N-glycan analysis of a protein derived from an IF fraction, usually secreted proteins are analysed from total protein fractions. This might be a reason why less heterogeneity was found.

Evidently, the N-glycans present on sc-bFSH exhibit considerable structural aberration from its native counterpart, pituitary bFSH (Baenzinger and Green, 1988). As anticipated from known plant N-glycan structures, no N-glycans of the mammalian complex-type were found, neither b1,4 linked galactose nor terminal sialic acid. b(1,2) xylose and core a(1,3) fucose have never been found in mammals cells and they are considered potentially immunogenic structures (Wilson et al., 1998; Kurosoka et al., 1991; Faye et al., 1993??). Although so far no negative effect has been reported for plantibodies applied to mammals which might result from these sugars, no long term studies are available.

We showed evidence, that the plant-produced hormone has in vivo bioactivity.

As appointed above, another important aspect of these experiment is the fact that the application of a highly concentrated IF extract, which comprises a complex mixture of periplasmic proteins, did not have an deleterious effect on the model animal. Unlike other established protein expression systems, such as bacterial, yeast or animal cell culture systems, plant IF extracts may be directly applicable in acute medical treatments without the need of further expensive purification.

In summary we conclude that the TMV-based expression system provided here gives a very attractive expression system for mammalian glycoproteins such as glycoprotein hormones, since bioactive glycoforms of sc-bFSH accumulate to high levels in the periplasmic space of N. benthamiana leaves. We also could demonstrate the important benefit of being able to apply crude protein IF-extracts without the concerns of an exposure to potentially infectious agents and apparently without any acute deleterious effect to the model animal.

Abbreviations Used

GlcNAc, N-Acetylglucosamine; Fuc, fucose; Gal, galactose; GalT, beta 1,4-galactosyltransferase; RCA, Ricinus Cummunis Agglutinin;

Figure Legends

FIG. 1

Major differences between mammalian and plant complex N-linked glycans. Drawn are typical N-linked glycans. Numerous variations, both extended or truncated, occur in mammals and plants.

FIG. 2

Comparison of RNA levels and product of β1,4-galactosyltransferase. Upper panel: Northern blot of total RNA isolated from 25 transgenic plants, including a not transformed control plant (0), detected with a human β1,4-galactosyltransferase probe. Lower panel: Western blot of the same plant probed with RCA to detect terminal galactose residues on glycoproteins. M. indicates the molecular weight marker.

FIG. 3

Western blot showing the binding of lectin and antibody to protein isolated from wild-type and a β1,4-galactosyltransferase plant (no. 8 from FIG. 2). A: RCA as in FIG. 2, B: anti HRP (detecting both xylose and fucose) antibody, C: anti xylose antibody, D: anti fucose antibody.)

FIG. 4

Western blot showing RCA and sheep-anti-mouse-IgG binding to purified antibody produced in hybridoma culture (Hyb), tobacco plants (plant) and tobacco plants co-expressing β1,4-galactosyltransferase (GalT11 and GaIT12). H.C.: heavy chain, L.C. light chain.

FIG. 5

Western blot showing specific antibody binding to recombinant beta-FSH and recombinant alpha- and beta-FSH expressed in plants. Using an anti-human FSH beta subunit antiserum we could demonstrate the expression of the beta bFSH also in alpha/beta cotransfected plants. The beta-bFSH appeared as a double band at about 14 kDa.

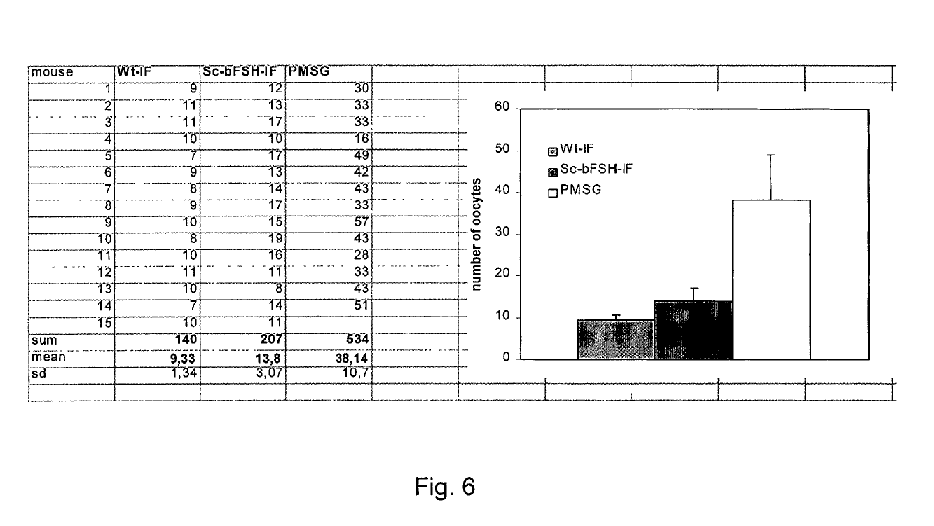

FIG. 6

In vivo bioassay for the determination of the activity of biopharmaceutical plant-derived glycoprotein hormone preparations in mice. Superovulatory treatment of C57/CBA mice with sc-bFSH: The responses, i.e. numbers of counted oocytes, of 15 or 14 mice treated each with either sc-bFSH IF extract corresponding to approx. 4.8 IU, or with equal amounts of IF extract of not infected wildtype plants (wt-IF, negative control) or with PMSG corresponding to each 5 IU of FSH (positive control) are listed in the table. The total (sum) and mean numbers of oocytes including the standard deviations for the three groups are given. The diagram illustrates the mean numbers of oocytes counted for the three groups of mice treated with sc-bFSH-IF, wt-IF or PMSG. The standard deviations are indicated (SD). A, B:

### References

Aoki, D., Lee, N., Yamaguchi, N., Dubois, C., and Fukuda, M. N. (1992). Golgi retention of a trans-Golgi membrane protein, galactosyltransferase, requires cysteine and histidine residues within the membrane-anchoring domain. Proceedings Of The National Academy Of Sciences Of The United States Of America 89, 4319-4323.

Asano, M., Furukawa, K., Kido, M., Matsumoto, S., Umesaki, Y., Kochibe, N., and Iwakura, Y. (1997). Growth retardation and early death of beta- 1,4-galactosyltransferase knockout mice with augmented proliferation and abnormal differentiation of epithelial cells. Embo j 16, 1850-7.

Boyd, P. N., Lines A. C., and Patel A. K. (1995). The effect of the removal of sialic acid, galactose and total carbohydrate on the functional activity of Campath-1H. Mol immunol 32, 1311-8.

Cabanes Macheteau, M., Fichette Laine, A. C., Loutelier Bourhis, C., Lange, C., Vine, N. D., Ma, J. K., Lerouge, P., and Faye, L. (1999). N-Glycosylation of a mouse IgG expressed in transgenic tobacco plants. Glycobiology 9, 365-72.

De Vries, S., Hoge, H., and Bisseling, T. (1991). Isolation of total and polysomal RNA from plant tissues. In Plant Molecular Biology Manual, B. Gelvin, R. A. Schilperoort and D. P. S. Verma, eds. (Dordrecht: Kluwer Academic Publishers), pp. B6/1-13.

Dieryck, W., Pagnier J., Poyart, C., Marden, M. C., Gruber, V., Bournat, P., Baudino, S., and Merot, B. (1997). Human haemoglobin from transgenic tobacco [letter] Nature 386, 29-30.

Faye, L., Gomord, V., Fitchette Laine, A. C. and Chrispeels, M. J. (1993). Affinity purification of antibodies specific for Asn-linked glycans containing alpha 1→3 fucose or beta 1→2 xylose. Anal Biochem 209, 104-8.

Fichette Laine, A. C., Gomord, V., Cabanes, M., Michalski, J. C., Saint Macary, M., Foucher, B., Cavelier, B., Hawes, C., Lerouge, P., and Faye, L. (1997). N-glycans harboring the Lewis a epitope are expressed at the surface of plant cells. Plant J 12, 1411-7.

Florack, D., Allefs, S., Bollen, R., Bosch, D., Visser, B., and Stiekema, W. (1995). Expression of giant silkmoth cecropin B genes in tobacco. Transgenic Research 4, 132-141.

Herman, T., and Horvitz, H. R. (1999). Three proteins involved in Caenorhabditis elegans vulval invagination are similar to components of a glycosylation pathway. Proc Natl Acid Sci U S A 96, 979-9.

Hollister, J. R., Shaper, J. H. and Jarvis, D. L. (1998). Stable expression of mammalian beta, 1,4-galactosyltransferase extends the N-glycosylation pathway in insect cells. Glycobiology 8, 473-80.

Horsch, R. B., Fry, J. E., Hoffmann, N. L., Eichholtz, D., Rogers, S. G., and Fraley, R. T. (1985). A simple and general method for transferring genes into plants. Science, USA 227, 1229-1231.

Loffe, E., and Stanley, P. (1994). Mice lacking N-acetylglucosaminyltransferase I activity die at mid-gestation, revealing and essential role for complex or hybrid N-linked carbohydrates. Proc Natl Acid Sci U S A 91, 728-32.

Jarvis, D. L., and Finn, E. E. (1996). Modifying the insect cell N-glycosylation pathway with immediate early baculovirus expression vectors. Nat Biotechnol 14, 1288-92.

Jenkins, N. Parekh, R. B., and James D. C. (1996). Getting the glycosylation right: implications for the biotechnology industry. Nat Biotechnol 14, 975-81.

Johnson, K. D., and Chrispeels, M. J. (1987). Substrate specificities of N-acetylglucosaminyl-, fucosyl-, and xylosyltransferases that modify glycoproteins in the Golgi apparatus of bean cotyledons. Plant Physiology 84, 1301-1308.

Lerouge, P., Cabanes Macheteau, M., Rayon, C, Fischette Laine, A. C., Gomord, V, and Faye, L. (1998). N- glycoprotein biosynthesis in plants: recent developments and future trends. Plant Mol Biol 38, 31-48.

Ma, J. K., Hiatt, A., Hein, M., Vine, N. D., Wang, F., Stabila, P., van Dolleweerd, C., Mostov, K. and Lehner, T. (1995). Generation and assembly of secretory antibodies in plants [see comments.] Science 268, 716-9.

Masri, K. A., Appert, H. E., and Fukuda, M. N. (1988). Identification of the full-length coding sequence for human galactosyltransferase (beta-N-acetylglucosaminide: beta 1,4-galactosyltransferase). Biochem Biophys Res Commun 157, 657-63.

Matsumoto, S., Ikura, K., Ueda, M., and Sasaki, R. (1163). Characterization of a human glycoprotein (erythropoietin) produced in cultured tobacco cells. Plant Molecular Biology 27, 1163-1172.

Melo, N. S., Nimtz, M., Conradt, H. S., Fevereiro, P. S., and Costa, J. (1997). Identification of the human Lewis(a) carbohydrate motif in a secretory peroxidase from a plant cell suspension culture (Vaccinium myrtillus L.). FEBS Lett 415, 186-91.

Palacpac, N. Q., Kimura, Y., Fuijyama, K., Yoshida, T., and Seki, T. (1999). Structures of N-linked oligosaccharides of glycoproteins from tobacco BY2 suspension cultured cells. Biosci Biotechnol Biochem 63, 35-9.

Palacpac, N. Q., Yoshida, S., Sakai, H., Kimura, Y., Fuijyama, K., Yoshida, T., and Seki, T. (1999). Stable expression of human beta 1,4-galactosyltransferase in plant cells modifies N-linked glycosylation patterns. Proc Natl Acad Sci U S A 96, 4692-7.

Rayon, C., Cabanes Macheteau, M., Loutelier Bourhis, C., Salliot Maire, I., Lemoine, J., Reiter, W. D. Lerouge, P., and Faye, L. (1999). Characterization of N-glycans from Arabidopsis. Application to a fucose-deficient mutant. Plant Physiol 119, 725-34.

Saito, K., Noji, M. Ohmori, S., Imai, Y., and Murakoshi, I. (1991). Integration and expression of a rabbit liver cytochrome P-450 gene in transgenetic Nicotiana tabacum. Proceedings Of The National Academy Of Sciences Of The United States Of America 88, 7041-7045.

Sambrook, J., Fritsch, E. F., and Maniatis, T. (1989). Molecular Cloning: A Laboratory Manual (Plainview, N.Y.: Cold Spring Harbor Lab. Press).

Schachter, H. (1991). The ‘yellow brick road’ to branched complex N-glycans. Glycobiology 1, 453-61.

Schindler, T., Bergfeld, R., and Schopfer, P. (1995). Arabinogalactan proteins in maize coleoptiles: developmental relationship to cell death during xylem differentiation but not to extention growth. Plant JU 7, 25-36.

Schaper, N. L., Shaper, J. H., Meuth, J. L., Fox, J. L., Chang, H., Kirsch, I. R. and Hollis, G. F. (1986). Bovine galactosyltransferase: identification of a clone by direct immunological screening of a cDNA expression library. Proc Natl Acad Sci U S A 83, 1573-7.

Smant, G., Goverse, A., Stokkermans, J. P. W. G., De Boer, J. M., Pomp, H., Zilverentant, J. F. Overmars, H. A. Helder, J., Schots, A. and Baaker, J. (1997) Potato root diffusate-induced secretion of soluble, basic proteins originating from the subventral esophageal glands of potato cyst nematodes. Phytopathology 87, 839-845.

Stanley, P., and loffe, E, (1995). Glycosyltransferase mutants: key to new insights in glycobiology. Faseb j 9, 1436-44.

Stanley, P., Raju, T. S., and Bhaumik, M. (1996). CHO cells provide access to novel N-glycans and developmentally regulated glycosyltransferases. Glycobiology 6, 695-9.

Thanavala, Y., Yang, Y. F., Lyons, P., Mason, H. S., and Arntzen, C. (1995). Immunogenicity of transgenetic plant-derived hepatitis B surface antigen. Proceedings of the National Academy of Sciences of the United States of America 92, 3358-3361.

van Engelen, F. A., Molthoff, J. W., Conner, A. J., Nap, J. P., Pereira, A., and Stiekema, W. J. (1995). pBINPLUS: an improved plant transformation vector based on pBIN19. Transgenetic Res 4, 288-90.

van Engelen, F. A., Schouten, A., Molthoff, J. W., Roosein, J. Salinas, J., Dirkse, W. G., Schots, A., Bakker, J., Gommers, F. J., Jongsma, M. A., and et al. (1994). Coordinate expression of antibody subunit genes yields high levels of functional antibodies in roots of transgenetic tobacco. Plant Mol Biol. 26, 1701-10.

von Schaewen, A., Sturm, A., O'Neill, J., and Chrispeels, M. J. (1993) Isolation of a mutant Arabidopsis plant that lacks N-acetyl glucosaminyl transferase I and is unable to synthesize Golgi-modified complex N-linked glycans. Plant Physiol 102, 1109-18.

Yamaguchi, N., and Fukuda, M. N. (1995). Golgi retention mechanism of beta-1,4-galactosyltransferase. Membrane-spanning domain-dependent homodimerization and association with alpha- and beta-tubulins. J Biol Chem 270, 12170-6.

Abdennebi L, Couture L, Grebert D, Pajot E, Salesse R, Remy J J. Generating FSH antagonists and agonists through immunization against FSH receptor N-terminal decapeptides. J Mol Endocrinol. 1999 April;22(2):151-9.

Altmann F, Staudacher E, Wilson I B and Marz. Insect cells as hosts for the expression of recombinant glycoproteins. Glycoconj J. 1999 February;16(2):109-23.

Baenzinger J U, Green E D. Pituitary glycoproteins hormone oligosaccharides: structure, synthesis and function of the asparagine linked oligosaccharides on lutropin, follitropin and thyrotropin. Biochim. Biophys. Acta 1988 947:287-306.

Bielinska M and Boime I. The glycoprotein hormone family: structure and function of the carbohydrate chains. In Glycoproteins. Montreuil J, Schachter H and Vliegnhart J F G (Eds.). Elsevier Science 1995. Chapter 11:565-578

Boevink P, Oparka K, Santa Cruz S, Martin B, Betteridge A, Hawes C. 1998. Stacks on tracks: the plant Golgi apparatus traffics on an actin/ER network. Plant J. 15:441-7.

Cabanes-Macheteau M, Fitchette-Laine A C; Loutelier-Bourhis C, Lange C, Vine N D, Ma J K, Lerouge P, Faye L. N-Glycosylation of a mouse IgG expressed in transgenic tobacco plants. Glycobiology. 1999 April; 9(4):365-72.

Carlsson S R. Isolation and characterization of glycoproteins. In Glycobiology. A Practical Aproach. IRL Press. Oxford UK 1993 pp1-26

Casper S J, Holt C A. Expression of the green fluorescent protein-encoding gene from a tobacco mosaic virus-based vector. Gene. 1996; 173:69-73.

Chappel S C. Heterogeneity of follicle stimulating hormone: control and physiological function. Hum Reprod Update. 1995 September;1(5):479-87.

Chrispeels M J and Faye L. The production of recombinant glycoproteins with defined non-immunogenic glycans. In: Transgenic plants: a production system for industrial and pharmaceutical proteins. Owen M R L, Pen J, (eds). John Wiley, Chichester, UK 1996 pp. 99-113.

Cockett M I, Bebbington C R, Yarranton G T. High level expression of tissue inhibitor of metalloproteinases in Chinese hamster ovary cells using glutamine synthetase gene amplification. Biotechnology (N.Y.). 1990 July;8(7):662-7.

Drickamer K. Clearing up glycoprotein hormones. Cell. Dec. 20, 1991; 67(6):1029-32.

Essl D, Dirnberger D, Gomord V, Strasser R, Faye L, Glossl J, Steinkellner H. The N-terminal 77 amino acids from tobacco N-acetylglucosaminyltransferase I are sufficient to retain a reporter protein in the Golgi apparatus of Nicotiana benthamiana cells. FEBS Lett. Jun. 18, 1999; 453(1-2):169-73.

Faye L, Gomord V, Fitchette-Laine A C, Chrispeels M J. Affinity purification of antibodies specific for Asn-linked glycans containing alpha 1→3 fucose or beta 1→2 xylose. Anal Biochem. Feb. 15, 1993; 209(1):104-8.

Feng W, Matzuk M M, Mountjoy K, Bedows E, Ruddon R W, Boime I. The asparagine-linked oligosaccharides of the human chorionic gonadotrophin beta subunit facilitate correct disulfide bond pairing. J Biol Chem May 19, 1995; 270(20): 11851-9

Flanders D J, Rawlins D J, Shaw P J, Lloyd C W. Nucleus-associated microtubules help determine the division plane of plant epidermal cells: avoidance of four-way junctions and the role of cell geometry. J Cell Biol. 1990 April; 110(4):1111-22.

Goodbody K C and LLoyd C W 1994. Immunofluorescence techniques for analysis of the cytoskeleton. In Plant Cell Biology. A Practical Approach. Harris H and Oparka K J. eds. IRL Press. Oxford University Press. Oxford, New York, Tokyo. 1994 pp 221-243

Hiatt A, Cafferkey R, Bowdish K. Production of antibodies in transgenic plants. Nature. Nov. 2, 1989; 342(6245):76-8.

Horton M H. In Vitro Recombination and Mutagenesis of DNA. In Methods in Molecular Biology 15. PCR protocols. Current Methods and Applications. White BA ed. Humana Press Inc., Totowa, N.J. 1993 pp. 251-261

Jensen O N, Shevchenko A and Mann M. Protein analysis by mass spectrometry. In Protein Structure. A Practical Approach. Creighton T E ed. IRL Press. Oxford University Press. Oxford, New York, Tokyo. 1997 pp 29-57.

Kolarich D, Altmann F. N-glycan analysis by matrix assisted laser desorption/ionisation mass spectrometry of electrophoretically separated non-mammalian proteins. Application to peanut allergen Ara h 1 and olive pollen allergen Ole e 1. Anal. Biochem. (in press)

Krebitz M, Wiedermann U, Essl D, Steinkellner H, Wagner B, Turpen T H, Ebner C, Scheiner O, Breiteneder H. 2000. Rapid production of the major birch pollen allergen Bet v 1 in Nicotiana benthamiana plants and its immunological in vitro and in vivo characterization. FASEB J. 14:1279-1288.

Kurosaka A, Yano A, Itoh N, Kuroda Y, Nakagawa T, Kawasaki T. The structure of a neural specific carbohydrate epitope of horseradish peroxidase recognized by anti-horseradish peroxidase antiserum. J Biol Chem. Mar 5, 1991; 266(7):4168-72. Kusnadi A R, Nikolov Z L and Howard J A. Production of recombinant proteins in transgenic plants: practical considerations. Biotechnology and Bioengineering 1997. 56: 473-484.

Laemmli U K. Cleavage of structural proteins during the assembly of the head of bacteriophage T4.Nature. Aug. 15, 1970; 227(259):680-5

Leiter H, Mucha J, Staudacher E, Grimm R, Glossl J, Altmann F. Nucleotide Purification, cDNA cloning, and expression of GDP-L-Fuc:Asn-linked GlcNAc alpha 1,3-fucosyltransferase from mung beans. J Biol Chem. 1999 274(31):21830-9.

Lerouge P, Cabanes-Macheteau M, Rayon C, Fischette-Laine A C, Gomord V, Faye L. N-glycoprotein biosynthesis in plants: recent developments and future trends. Plant Mol Biol. 1998 38(1-2):31-48.

Leung K, Kaynard A H, Negrini B P, Kim K E, Maurer R A, Landefeld T D. Differential regulation of gonadotrophin subunit messenger ribonucleic acids by gonadotrophin-releasing hormone pulse frequency in ewes. Mol Endocrinol. 1987 1(10):724-8.

Ma J K, Hiatt A, Hein M, Vine N D, Wang F, Stabila P, van Dolleweerd C, Mostov K, Lehner T. Generation and assembly of secretory antibodies in plants. Science. 1995 268(5211):716-9.

Ma J K, Hikmat B Y, Wycoff K, Vine ND, Chargelegue D, Yu L, Hein M B, Lehner T. 1998 Characterization of a recombinant plant monoclonal secretory antibody and preventive immunotherapy in humans. Nat Med. 4(5):601-6.

Maurer R A, Beck A. Isolation and nucleotide sequence analysis of a cloned cDNA encoding the beta-subunit of bovine follicle-stimulating hormone DNA. 1986 5(5):363-9.

Melo N S, Nimtz M, Conradt H S, Fevereiro P S, Costa J. Identification of the human Lewis(a) carbohydrate motif in a secretory peroxidase from a plant cell suspension culture (Vaccinium myrtillus L.). FEBS Lett. 1997 415(2):186-91.

Ogawa H, Hijikata A, Amano M, Kojima K, Fukushima H, Ishizuka I, Kurihara Y, Matsumoto I. Structures and contribution to the antigenicity of oligosaccharides of Japanese cedar (Cryptomeria japonica) pollen allergen Cry j I: relationship between the structures and antigenic epitopes of plant N-linked complex-type glycans. Glycoconj J. 1996 13(4):555-66.

Palacpac N Q, Yoshida S, Sakai H, Kimura Y, Fujiyama K, Yoshida T, Seki T. Stable expression of human beta1,4-galactosyltransferase in plant cells modifies N-linked glycosylation patterns. Proc Natl Acad Sci U S A. 1999 96:4692-7.

Pierce J G, Parsons T F. Glycoprotein hormones: structure and function. Annu Rev Biochem. 1981;50:465-95.

*Pogue G P, Lindbo J A, Dawson W O and Turpen T H. Tobamovirus Transient Expression Vectors: Tools for Plant Biology and High-Level Expression of Foreign Proteins in Plants. In: Molecular Microbial Ecology Manual. Kluiwer Academic Publishers. The Netherlands. 1997

Remy J J, Lahbib-Mansais Y, Yerle M, Bozon V, Couture L, Pajot E, Greber D, Salesse R. The porcine follitropin receptor: cDNA cloning, functional expression and chromosomal localization of the gene. Gene. Oct. 3, 1995; 163(2):257-61.

Shevehenko A, Wilm M, Vorm O, Mann M. Mass spectrometric sequencing of proteins silver-stained polyacrylamide gels. Anal Chem. Mar 1, 1996; 68(5):850-8.

Shi Q and Jackowski G. One-dimensional polyacrylamide gel electrophoresis. In Gel Electrophoresis of Proteins. A Practical Approach. Hames B D ed. IRL Press. Oxford University Press. Oxford, New York, Tokyo. 1998 221-243

Strasser R, Mucha J, Schwihla H, Altmann F, Glossl J, Steinkellner H. Molecular cloning and characterization of cDNA coding for beta 1,2N-acetylglucosaminyltransferase I (GlcNAc-TI) from Nicotiana tabacum. Glycobiology. 1999 August;9(8):779-85.

Strasser R, Mucha J, Mach L, Altmann F, Wilson I B, Glossl J, Steinkellner H. Molecular cloning and functional expression of beta 1,2-xylosyltransferase cDNA from Arabidopsis thaliana.FEBS Lett. 2000 472(1): 105-8.

Strasser R, Steinkellner H, Borén M, Altmann F, Mach L, Glössl J, Mucha J. Molecular cloning of cDNA encoding N-acetylglucosaminyltransferase II from Arabidopsis thaliana. Glycoconj J. 16 787-791.

Sturm A. N-Glycosylation in plants proteins. In Glycoproteins. Montreuil J, Schachter H and Vliegenhart J F G (Eds.) Elsevier Science B.V. 1995. Chapter 9, 521-541

Sugahara T, Sato A, Kudo M, Ben-Menahem D, Pixley M R, Hsueh A J, Boime I. Expression of biologically active fusion genes encoding the common alpha subunit and the follicle-stimulating hormone beta subunit. Role of a linker sequence. J Biol Chem. May 3, 1996; 271(18):10445-8.

Suganuma N, Matzuk M M, Boime I. Elimination of disulfide bonds affects assembly and secretion of the human chorionic gonadotrophin beta subunit. J Biol Chem. Nov. 15, 1989;264(32):19302-7.

Tacket C O, Mason H S, Losonsky G, Estes M K, Levine M M, Arntzen C J. 2000 Human immune responses to a novel norwalk virus vaccine delivered in transgenic potatoes. J Infect Dis. 182:302-5.

Taticek R A, Lee C W, Shuler M L. Large-scale insect and plant cell culture. Curr Opin Biotechnol. 1994 February;5(2):165-74

Tezuka K, Hayashi M, Ishihara H, Nishimura M, Onozaki K, Takahashi N. Purification and substrate specificity of beta-xylosidase from sycamore cell (Acer pseudoplatanus L.): application for structural analysis of xylose-containing N-linked oligosaccharides. Anal Biochem. 1993 June; 211(2):205-9.

Tretter V, Altmann F, Marz L. Peptide-N4-(N-acetyl-beta-glucosaminyl)asparagine amidase F cannot release glycans with fucose attached alpha 1 . . . 3 to the asparagine-linked N-acetylglucosamine residue. Eur J Biochem. Aug 1, 1991; 199(3):647-52.

Ulloa-Aguirre A, Midgley A R Jr, Beitins IZ, Padmanabhan V. Follicle-stimulating isohormones: characterization and physiological relevance. Endocr Rev. 1995 December; 16(6):765-87

van de Wiel D F, van Rijn P A, Meloen R H, Moormann R J. High-level expression of biologically active recombinant bovine follicle stimulating hormone in a baculovirus system. J Mol Endocrinol. 1998February; 20(1):83-98.

Wee E G, Sherrier D J, Prime T A, and Dupree P. Targeting of active sialyltransferase to the plant Golgi apparatus. Plant Cell 10(10):1759-1768, 1998.

Whitelam G C, Cockburn B, Gandecha A R, Owen M R. Heterologous protein production in transgenic plants. Biotechnol Genet Eng Rev. 1993; 11: 1-29.

Wilson I B, Harthill J E, Mullin N P, Ashford D A, Altmann F. Core alpha 1,3-fucose is a key part of the epitope recognized by antibodies reacting against plant N-linked oligosaccharides and is present in a wide variety of plant extracts. Glycobiology. 1998 July;8(7):651-61.

Wilson J M, Jones A L, Moore K, Looney C R and Bondioli K R. 1993. Superovulation of cattle with a recombinant-DNA bovine follicle stimulating hormone. Animal Reprod Science 1993 33: 71-82.

Wu J B, Stanton P G, Robertson D M, Hearn M T. Isolation of FSH from bovine pituitary glands. J Endocrinol. 1993 April; 137(1):59-68.

1. A plant comprising a cell comprising a functional mammalian enzyme or functional fragment thereof providing N-glycan biosynthesis additionally having been provided with an expression vector comprising a nucleic acid encoding a thyroid-stimulating hormone or gonadotrophin or gonadotrophin-receptor or functional fragment thereof

2. A plant according to claim 1 wherein said enzyme comprises human β1,4-galactosyltransferase.

3. A plant according to claim 1 or 2 wherein said thyroid-stimulating hormone or gonadotrophin or gonadotrophin-receptor or functional fragment thereof comprises an extended N-linked glycan.

4. A plant according to claim 3 wherein a said extended N-linked glycan comprises galactose.

5. A plant according to claim 4 wherein a said extended N-linked glycan is devoid of xylose.

6. A plant according to claim 4 wherein a said extended N-linked glycan is devoid of fucose.

7. A plant according to anyone of claims 1 to 6 wherein said expression vector is derived from a plant virus.

8. A plant according to claim 7 wherein said virus is a tobamovirus such as tobacco mosaic virus.

9. A plant according to anyone of claims 1 to 8 which comprises a tobacco plant.

10. A plant according to anyone of claims 1 to 9 wherein said gonadotrophin comprises FSH.

11. Use of a plant according to anyone of claims 1 to 10 to produce a desired thyroid-stimulating hormone or gonadotrophin or gonadotrophin-receptor or functional fragment thereof.

12. Use according to claim 11 wherein said thyroid-stimulating hormone or gonadotrophin or gonadotrophin-receptor or functional fragment thereof comprises an extended N-linked glycan et least comprising galactose.

13. A method for obtaining a desired thyroid-stimulating hormone or gonadotrophin or gonadotrophin-receptor or functional fragment thereof comprising cultivating a plant according to anyone of claims 1 to 10 until said plant has reached a harvestable stage, harvesting and fractionating said plant to obtain fractionated plant material and at least partly isolating said thyroid-stimulating hormone or gonadotrophin or gonadotrophin-receptor from said fractionated plant material.

14. A plant-derived thyroid-stimulating hormone or gonadotrophin or gonadotrophin-receptor or functional fragment thereof comprising an extended N-linked glycan at least comprising galactose.

15. A plant-derived thyroid-stimulating hormone or gonadotrophin or gonadotrophin-receptor or functional fragment thereof obtained by a method according to claim 13.

16. Use of a thyroid-stimulating hormone or gonadotrophin or gonadotrophin-receptor or functional fragment thereof according to claim 14 or 15 for the production of a pharmaceutical composition.

17. Use of a gonadotrophin or gonadotrophin-receptor or functional fragment thereof according to claim 14 or 15 for the production of a pharmaceutical composition for the treatment of a reproductive disorder.

18. A pharmaceutical composition comprising a thyroid-stimulating hormone or gonadotrophin or gonadotrophin-receptor or functional fragment thereof according to claim 14 or 15.


SEQUENCE LISTING (7 sequences)

SEQ ID NO: 1 - protein, 8 aa, Artificial Sequence
  FLAG epitope
DYKDDDDK

SEQ ID NO: 2 - DNA, 40 bp, Artificial Sequence
  amplification primer
ggaaatcaaagaatttcctgatggagagtttacaatgcag

SEQ ID NO: 3 - DNA, 34 bp, Artificial Sequence
  amplification primer
agctatgcatctattaggatttgtgataataaca

SEQ ID NO: 4 - DNA, 30 bp, Artificial Sequence
  amplification primer
atatgagtcgacatgaagtctgtccagttc

SEQ ID NO: 5 - DNA, 40 bp, Artificial Sequence
  amplification primer
ctccatcaggaaattctttgatttccctgaaggagcagta

SEQ ID NO: 6 - DNA, 22 bp, Artificial Sequence
  amplification primer
tttttcccttttttgttttccg

SEQ ID NO: 7 - DNA, 21 bp, Artificial Sequence
  amplification primer
gatgatgattcggaggctact